(19) United States
(12) Patent Application Publication
(10) Pub. No.: US 2011/0230564 A1
(43) Pub. Date: Sep. 22, 2011

(54) ANT4 INHIBITOR COMPOUNDS AND METHODS OF USE THEREOF

(75) Inventors: Naohiro Terada, Gainesville, FL (US); David A. Ostrov, Gainesville, FL (US)

(73) Assignee: UNIVERSITY OF FLORIDA RESEARCH FOUNDATION, GAINESVILLE, FL (US)

(21) Appl. No.: 13/056,978

(22) PCT Filed: Jul. 31, 2009
(86) PCT No.: PCT/US09/04427

§ 371 Date: Jun. 6, 2011

Related U.S. Application Data
(60) Provisional application No. 61/137,710, filed on Aug. 1, 2008.

(51) Int. Cl. A61K 31/121; A61P 15/16; A61P 35/00; G06G 7/48

(52) U.S. Cl. 514/675; 703/11

(57) ABSTRACT

The invention relates to methods for inducing male contraception and to methods of treating cell proliferation related disorders or diseases, such as cancer. The invention further relates to pharmaceutical compositions for inducing male contraception and for treating cell proliferation related disorders or diseases.

### RELATED APPLICATIONS

This application claims the benefit of U.S. provisional patent application Ser. No. 61/137,710 filed Aug. 1, 2008, which is incorporated herein by reference.

### STATEMENT OF RIGHTS TO INVENTIONS MADE UNDER FEDERALLY SPONSORED RESEARCH

This work was supported in part by a grant from the National Institutes of Health (Grant No. HD060474). The government has certain rights in the invention.

### BACKGROUND OF THE INVENTION

The adenine nucleotide translocase (Ant), also called ADP/ATP carrier (Aac), mediates the exchange of ADP and ATP across the inner mitochondrial membrane, thus playing an essential role in energy metabolism in eukaryotic cells (Klingenberg 1989, Nelson 1998, Fiore 1998). Under respiring conditions, ATP produced within the mitochondria is exported to the cytosol through Ant to support cellular activities. In exchange, ADP is imported to provide a substrate for the conversion of ADP to ATP by the ATP synthase. Ant is the most abundant protein on the mitochondrial inner membrane. It belongs to the mitochondrial carrier family that supports a variety of transport activities across the mitochondrial inner membrane (Fiore 1998, Palmieri 2000). A typical Ant molecule is comprised of roughly 300-320 amino acid residues that form six transmembrane helices. Although the functional unit has been proposed to be a homodimer acting as a gated pore that channels single molecules of ADP and ATP (Hackenberg 1980), a recent publication indicates that Ant is functional as a monomer on the mitochondrial membrane (Bamber 2007).

In multicellular organisms, differential Ant expression depends on tissue type, developmental stage, cell proliferation state, oxygen conditions, etc. Humans possess four distinct ANT isoforms, encoded by four genes. ANT1 (SLC25A4) is expressed primarily in the heart and skeletal muscle (Huizing 1998). ANT2 (SLC25A5) and ANT3 (SLC25A6) are expressed in all the somatic tissues ubiquitously; however, ANT2 is more significantly expressed in rapidly growing cells and is inducible by hypoxia, while ANT3 appears to be constitutively expressed (Stepien 1992, Lunardi 1992). Lastly, utilizing various approaches, we and others recently identified a novel member of the Ant family, Ant4 (SLC25A31, Aac4, SFEC) in both humans and mice (Rodic 2005, Dolce 2005, Kim 2007). Further, we have determined that Ant4 is expressed exclusively in testis (Rodic 2005).

The human ANT4 gene was predicted to encode a 315 amino acid protein, and share amino acid sequence homology with the other previously identified ANT proteins (73%, 71% and 72% overall amino acid identity to ANT1, ANT2 and ANT3, respectively). The gene also consists of three tandem repeats of an approximately 100 residue domain, each of which contains two transmembrane regions, a characteristic shared by all members of the solute carrier family (Belzacq 2002). When tagged-murine Ant4 protein was expressed in cell lines, it localized to mitochondria. Due to its amino acid conservation Ant4 was predicted to be a specific carrier of ADP and ATP. ANT4 contains the RRRNIMM sequence at the end of the 5^{th} α-helix trans-membrane domain, which is uniquely conserved in all the Ants (but not in the other solute carrier family proteins) and is essential for ADP/ATP transport activity (Pebay-Peyroula 2003). Indeed, Dolce et al. (2005) reported that ANT4 specifically exchanges ADP and ATP, but not other solutes, by an electrogenic antiport mechanism.

Inhibitors of ADP/ATP exchange by ANTs have previously been identified, including bongkrekic acid and carboxyatractyloside, but these drugs inhibit all ANTs in a non-specific manner. A particular concern for the use of these prototype drugs in clinics is therefore the inhibition of ANT1, which is known to cause mitochondrial myopathy and hypertrophic cardiomyopathy.

### BRIEF SUMMARY OF THE INVENTION

The invention provides compounds and compositions thereof useful in methods for inducing contraception in a subject or reducing or eliminating male meiotic spermatocytes (without damaging other cell types) in a subject, the method comprising administering to a subject in need thereof a therapeutically effective amount of a compound delineated herein (e.g., an adenine nucleotide translocase 4 (ANT4) inhibitor). The invention also relates to methods for identifying such compounds and compositions. The invention also provides compounds and compositions thereof useful in methods of modulating sperm motility.

The invention also provides compounds and compositions thereof useful in methods of treating a subject suffering from or susceptible to a cell proliferation related disorder or disease, the method comprising administering to a subject in need thereof a therapeutically effective amount of a compound delineated herein (e.g., an adenine nucleotide translocase 4 (ANT4) inhibitor), to thereby treat the subject suffering from or susceptible to a cell proliferation related disorder or disease. The invention also relates to methods for identifying such compounds and compositions. The invention also provides compounds and compositions thereof useful in methods of modulating cells and cell proliferation.

Family-planning organizations estimate that half of all conceptions are unplanned and half of the resulting pregnancies are undesired. This high rate of unintended pregnancy can be attributed to inadequate access to or use of contraceptives, or both. It is important to provide alternative choices for effective contraception to meet the needs of people with different ethnic, cultural, and religious values. Since male-directed contraceptive options are extremely limited, development of male contraceptives with safety, efficiency and cost-performance is particularly desired.

It was recently reported that ANT4 is exclusively expressed in male germ cells (Brower 2007). ANT4 expression is particularly high during male meiosis and is essential for progression of male meiosis. Targeted depletion of ANT4 in mice results in meiotic arrest of male germs cells and subsequent male infertility. Since exogenous inhibition of meiosis is considered one of the best approaches to develop male contraceptives, here we developed chemical compounds which may specifically target ANT4. Based on a predicted structural pocket unique to ANT4 at the ADP/ATP binding site, we identified that small molecules which may selectively inhibit ANT4 over other ANTs by a molecular docking approach. Based on the determined function of ANT4, we consider that the identified small molecules will inhibit male meiosis and fertility in vivo, thus leading to development of novel male contraceptives.

We believe that ANT4 is a suitable molecular target to develop male contraceptives for the following reasons: (i)ANT4 is essential for the progression of male meiosis; (ii) ANT4 is significantly different from somatic ANTs in it's structure, which is critical for developing selective inhibitors using small molecules. Indeed, ANT4 has a unique structural pocket at the ADP/ATP binding site, which is suitable for interactions with drug-like small molecules; (iii) ANT4 is exclusively expressed in the male germ cells, with no expression in any other tissue type. Thus, the chance that ANT4 selective inhibitors would cause deleterious side effects is minimal. Indeed, ANT4 knockout mice do not show any abnormality except male infertility.

Thus, the invention has considerable potential to identify lead compounds and ultimately develop novel male contraceptives that selectively eliminate male meiotic spermatocytes without damaging other cell types in the body.

The compounds and methods of use thereof include those delineated herein wherein the ANT4 inhibitor compound impairs transition during spermatogenesis from the leptotene stage to the zygotene stage in the progression of male meiosis.

In yet another aspect, the invention provides the use of a compound of any of the formulae herein alone or together with one or more of the herein-described second therapeutic agents in the manufacture of a medicament, either as a single composition or as separate dosage forms, for treatment or prevention in a subject of a disease, disorder or symptom set forth above, or for prevention of undesired pregnancies. Another aspect of the invention is a compound of the formulae herein for use in the treatment or prevention in a subject of a disease, disorder or symptom thereof delineated herein.

### BRIEF DESCRIPTION OF THE DRAWINGS

The present invention is further described below with reference to the following non-limiting examples and with reference to the following figures, in which:

FIG. 1 is an atomic homology model of mouse ANT4.

FIG. 2 illustrates the molecular surface of a homology modle of mouse ANT4 with positions that differ between ANT4 (blue) and other ANTs (gold).

FIG. 3 shows the amino acid sequences of various ANTs with the cleft site for molecular docking.

FIG. 4 shows various sequences of ANT1, ANT2, ANT3 and ANT4 with the 3^{rd} matrix loop region (M3) distinguished between ANT4 and the others.

FIG. 5 illustrates the results of mouse sperm staining with ANT4 antibodies.

FIG. 6 illustrates the results of various test compounds in modulating sperm motility.

FIG. 7 illustrates a testing protocol for test compound.

FIG. 8 illustrates the protocol and method of visualization of results of effects of compounds on male meiosis in vivo.

FIG. 9 illustrates defective spermatogenesis in Ant4-deficient mice.

FIG. 10 illustrates results of examination of meiotic arrest using Sycp3 staining in seminiferous tubule sections.

FIG. 11 illustrates the results of examination of meiotic arrest using germ cell chromosomal spread analysis.

FIG. 12 illustrates the expression profile of ANT4.

FIG. 13 illustrates the apoptic profile of adult testis in ANT-4 deficient mice.

### FIGURE LEGENDS

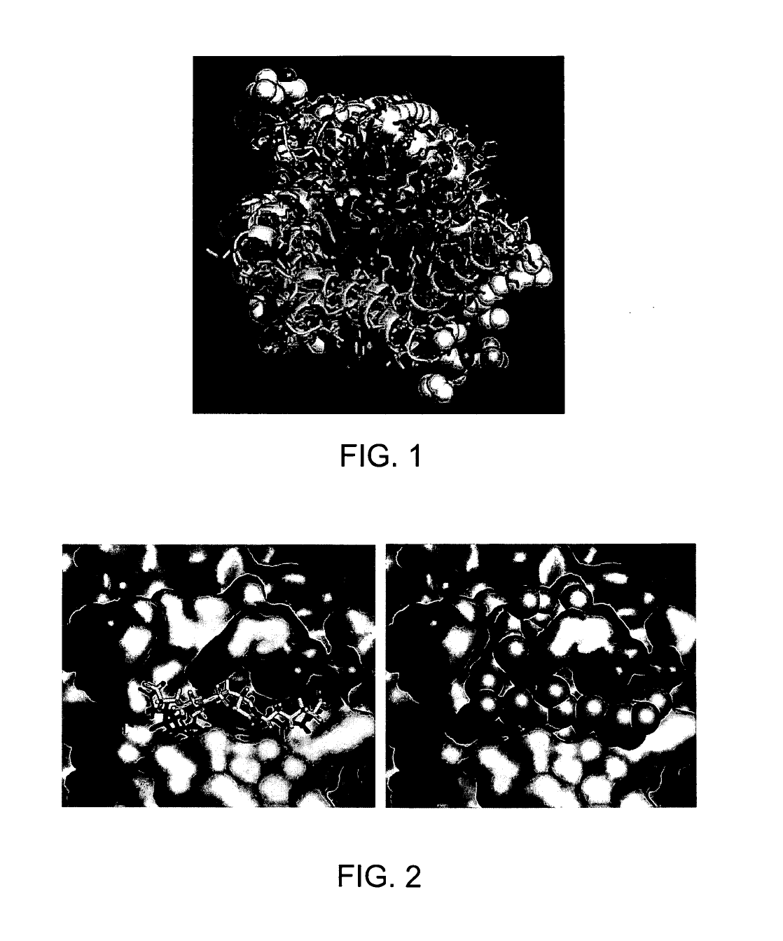

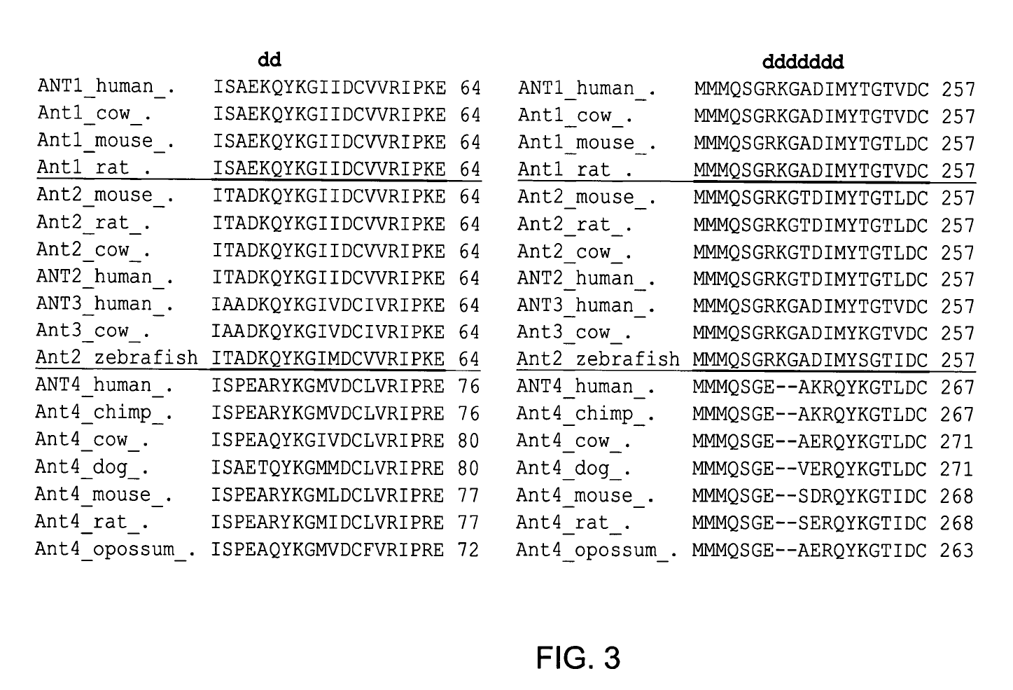

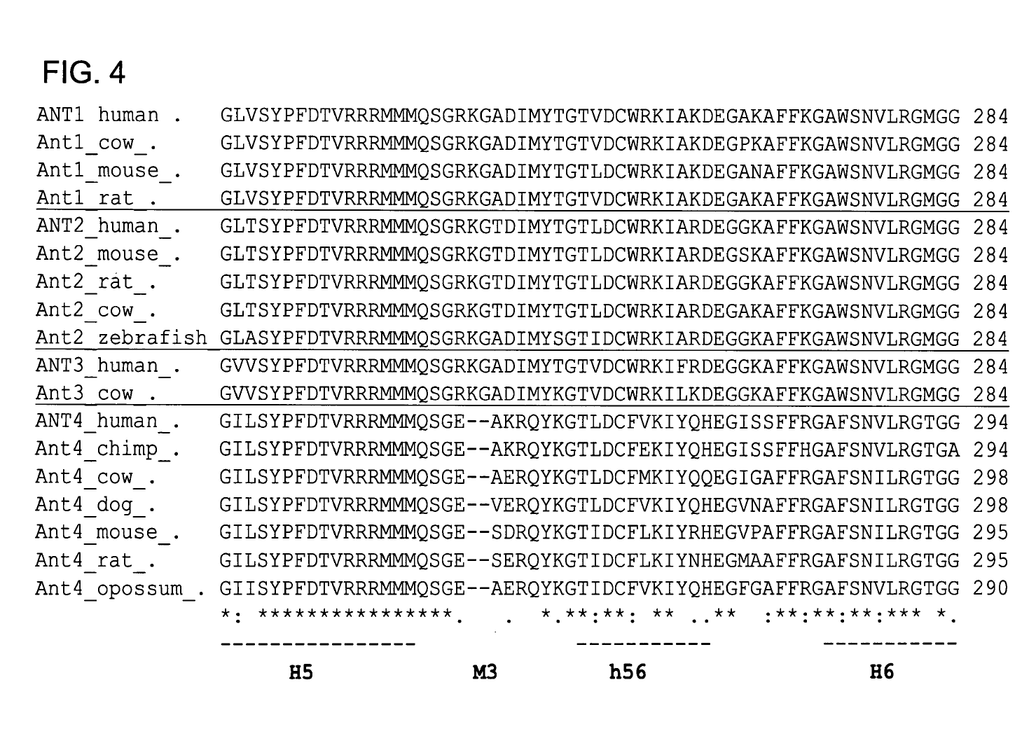

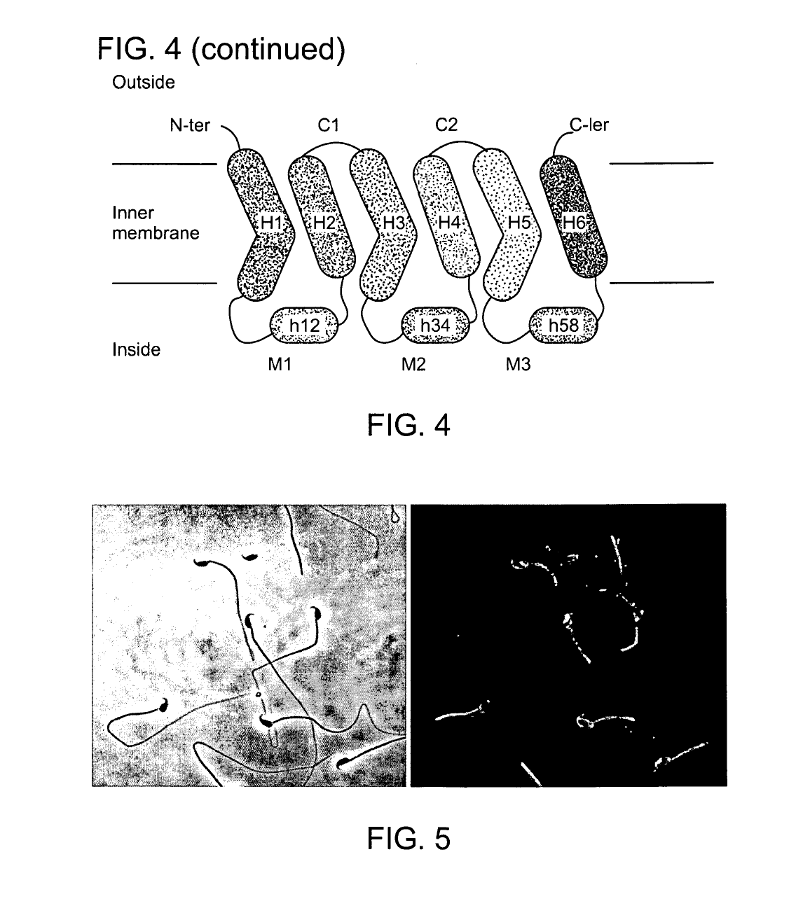

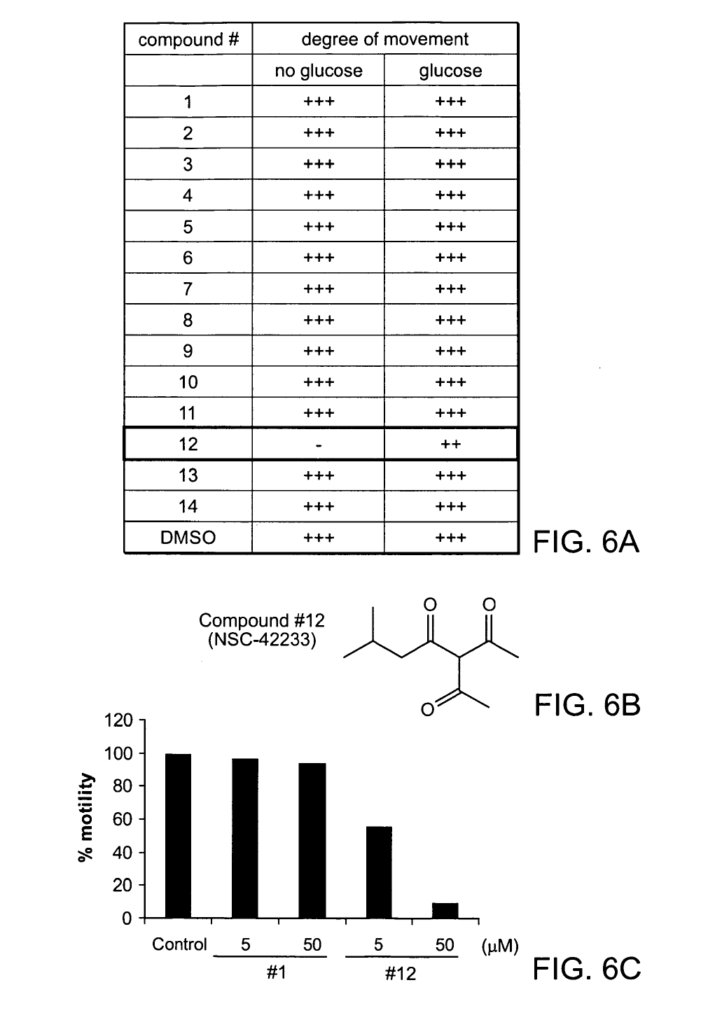

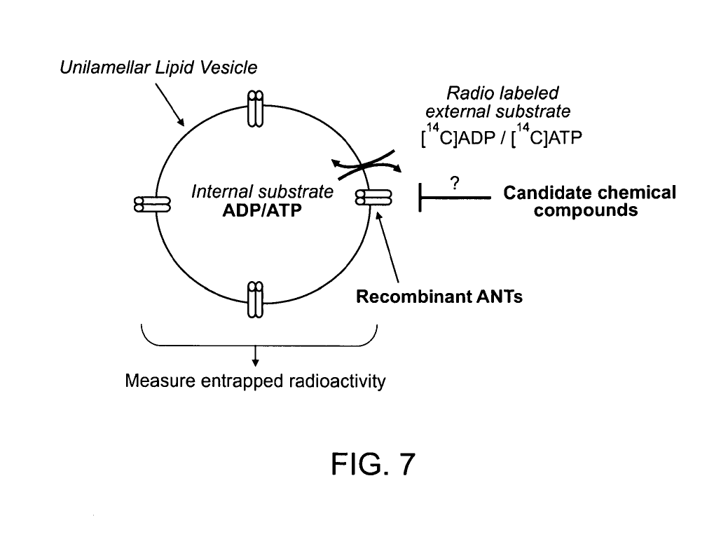

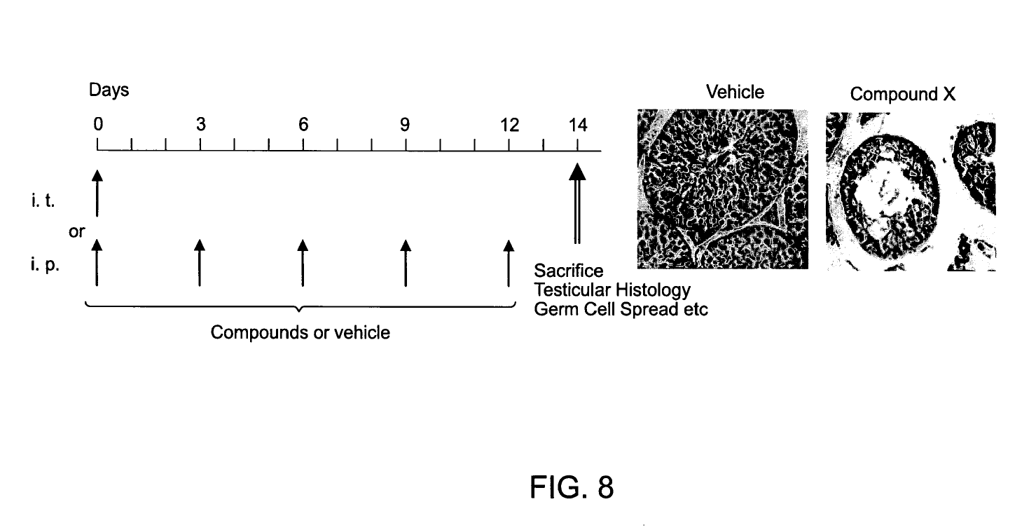

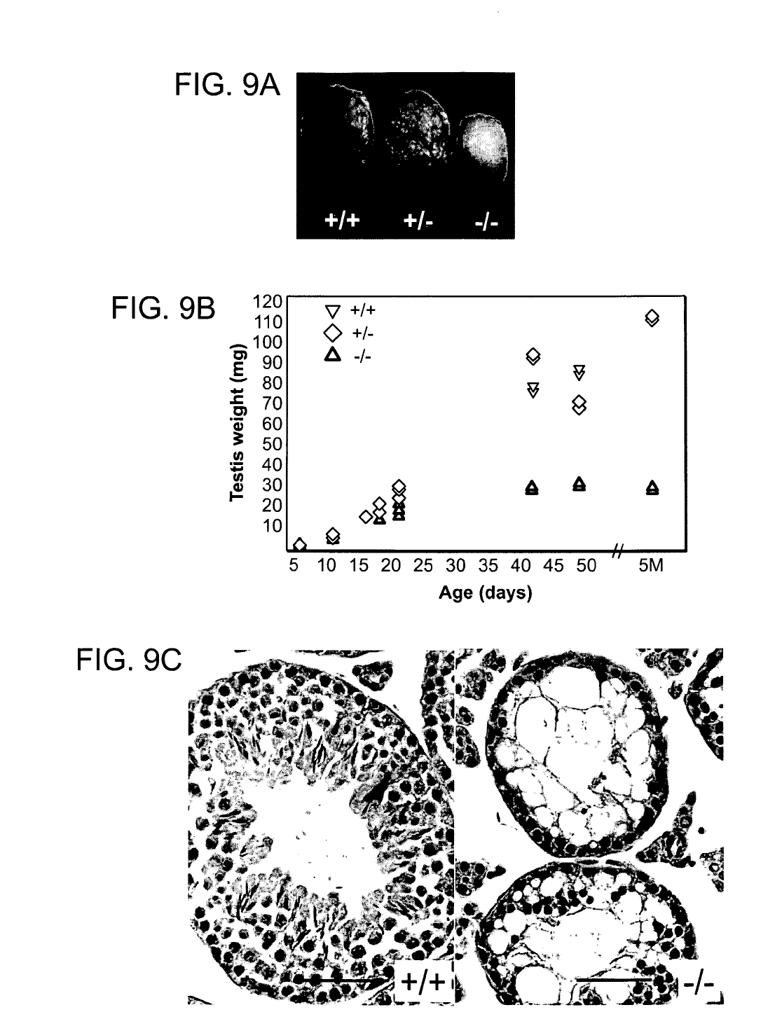

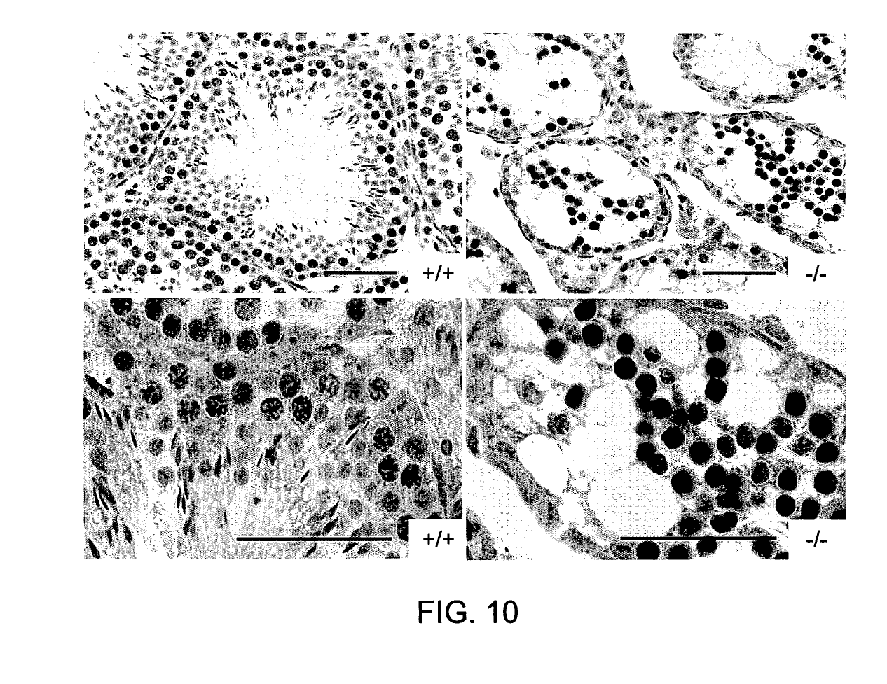

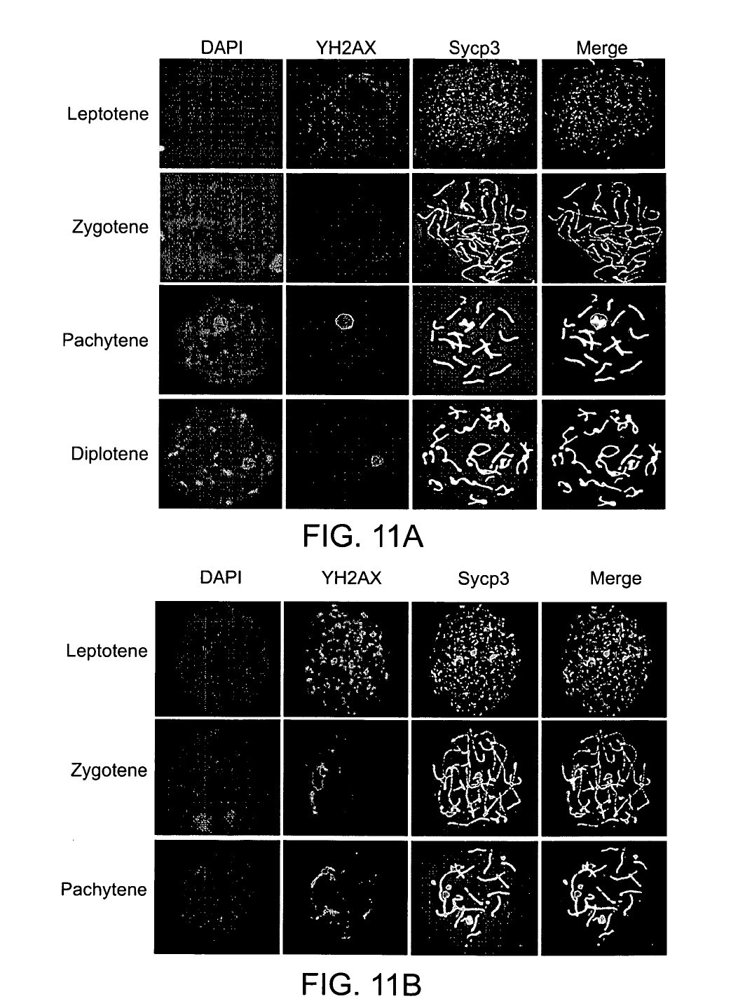

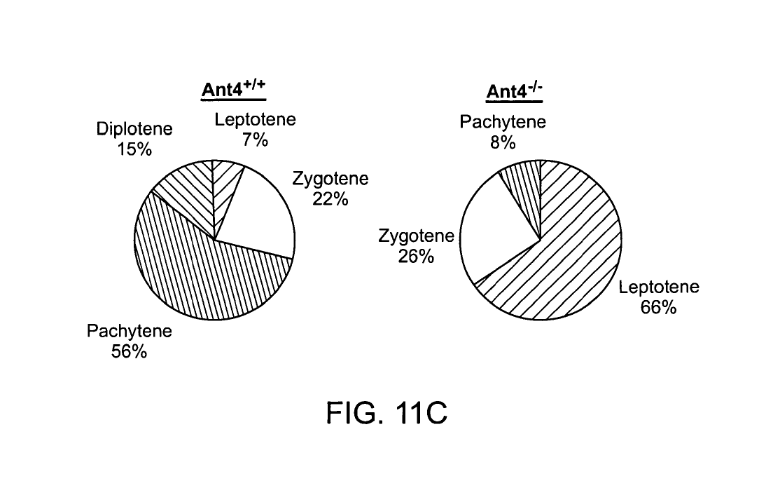

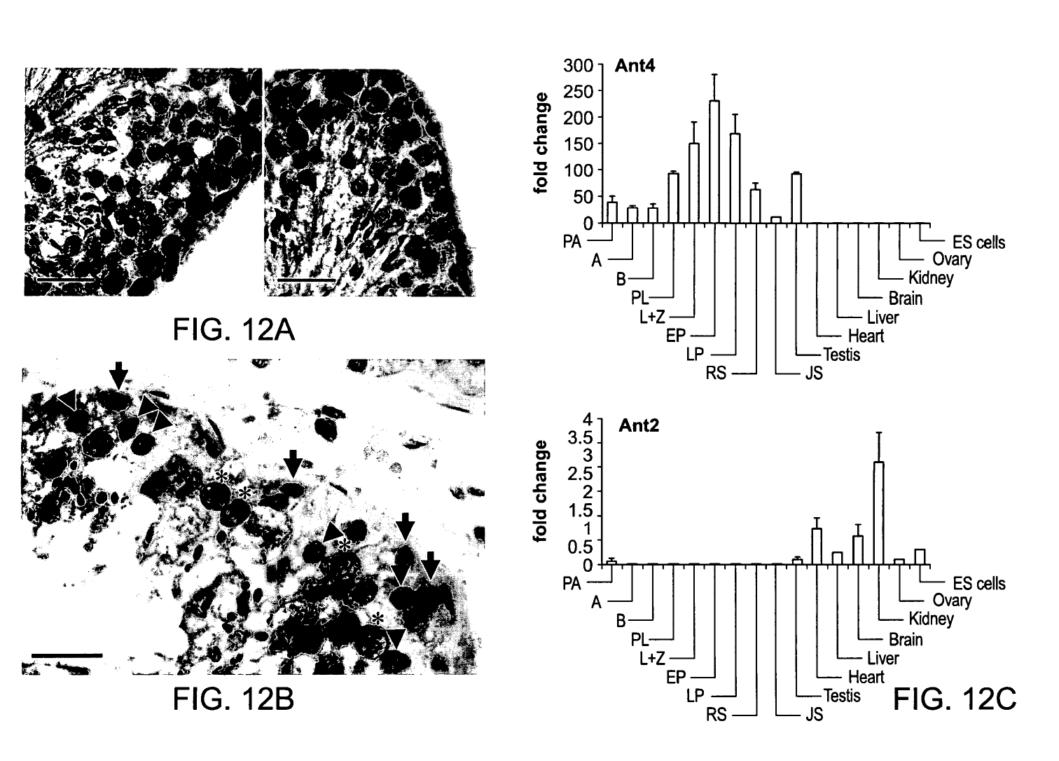

FIG. 1: Structure of mouse Ant4
An atomic homology model of mouse Ant4 is shown (in green) superimposed on the crystal structure of a bovine Ant1 (in magenta). Non-protein atoms are shown (yellow and red spheres) binding to Ant4 structural pockets that are suitable for interactions with drug-like small molecules.
FIG. 2: The site selected for molecular docking with a predicted high-scoring compound
The structural pocket unique to Ant4 is shown with a predicted high-scoring compound. Spheres in orange highlight amino acids uniquely conserved in Ant4 but not in other Ants (amino acids 62-63 & 257-261 of mouse Ant4, marked as “d” in FIG. 3).
FIG. 3: The selected site for molecular docking
The selected sites for molecular docking are marked with “d” above, which are conserved within Ant4, but different in Ant1, 2 & 3.
FIG. 4: ANT sequences
The sequences of various ANT family members are illustrated.
FIG. 5: ANT presence
The results of mouse sperm staining with ANT4 antibodies.
FIG. 6: Sperm motility
The results of various test compounds in modulating sperm motility.
FIG. 7: ADP/ATP exchange assay using proteoliposomes
Recombinant Ants are reconstituted on liposome membranes and the effects of the chemical compounds on radio-labeled ADP/ATP exchange through Ant4 and control Ant1 and Ant2 are measured.
FIG. 8: Effects of compounds on male meiosis in vivo
The selected compounds are injected either intratesticularly (i.t.) (one time at Day 0) or intraperitoneally (i.p.) (every 3 days) into 8 week old male C57B6 mice. The mice are sacrificed at 14 days and testicular histology examined. A representative potential outcome is shown using the images from wild type and Ant4 null testes.
FIG. 9: Defective spermatogenesis in Ant4-deficient mice
A. Gross morphology of testis from 6-week-old mice. B. Weight comparison of testis of the indicated genotypes (7 to 49 days old and 5 months). C. Histological analysis of testis (6-week-old) by hematoxylin and eosin staining. Scale bars: 50 μm.
FIG. 10: ANT Deficient cells
The results of stained testicular cross-sections with anti-synaptonemal complex protein 3 (Sycp3) antibody.
FIG. 11: Ant4-deficient mice exhibit meiotic arrest
A. Chromosomal spread analysis of freshly dissected testes from 6 week old wild-type mice. Spermatocytic preparations were incubated with both rabbit polyclonal Sycp3 (Ab15092, Abcam) and mouse monoclonal γH2AX (Ab22551, Abcam) at 1:200 dilutions. Sycp3 staining was visualized with an Alexa-fluor 488-conjugated anti-rabbit secondary antibody and γH2AX was visualized with a Cy3-conjugated anti-mouse secondary antibody. DAPI was added to slides to visualize DNA. B. Chromosomal spread analysis of freshly dissected testes from 6 week old Ant4-deficient mice. C. Percentage analysis of the spermatocytic cells present in the seminiferous epithelium of Ant4 wild-type and Ant4-deficient testes.
FIG. 12: Ant4 expression is highest in spermatocytes
A. Immunohistochemical analysis of Ant4 expression in mouse testis: Formalin-fixed, paraffin-embedded sections of mouse testis from wild-type 6-week-old mice were incubated with a rabbit polyclonal antibody against mouse Ant4. Ant4 staining was visualized using DAB (brown), and slides were counterstained with hematoxylin. In control (top left), rabbit IgG was used as a primary antibody. Scale bars: 40 μm. B. Immunohistochemical analysis of ANT4 expression in human testis: Formalin-fixed, paraffin-embedded sections of human testis from a 32 old male were incubated with a rabbit polyclonal antibody raised against human ANT4. ANT4 staining was visualized using DAB (brown), and slides were counterstained with hematoxylin. Arrows, arrowheads and asterisks indicate spermatogonia, Sertoli cells and spermatocytes, respectively. Scale bars: 50 μm. C. TaqmAnt^{M} Real-time PCR analysis of Ant4 and Ant2 transcript levels in purified mouse spermatogenic cell types (PA, primitive type A spermatogonia; A, type A spermatogonia; B, type B spermatogonia; PL, preleptotene spermatocyes; L+Z, leptotene+zygotene spermatocytes; EP, early pachytene spermatocytes; LP, late pachytene spermatocytes; RS, round spermatids; JS, juvenile Sertoli cells) and various other tissues (whole testis, heart, liver, brain, kidney, ovary, and embryonic stem cells) (6 week-old-mice). The relative transcript levels are shown in each graph when the transcript level of Ant4 and Ant2 in heart was set to 1.

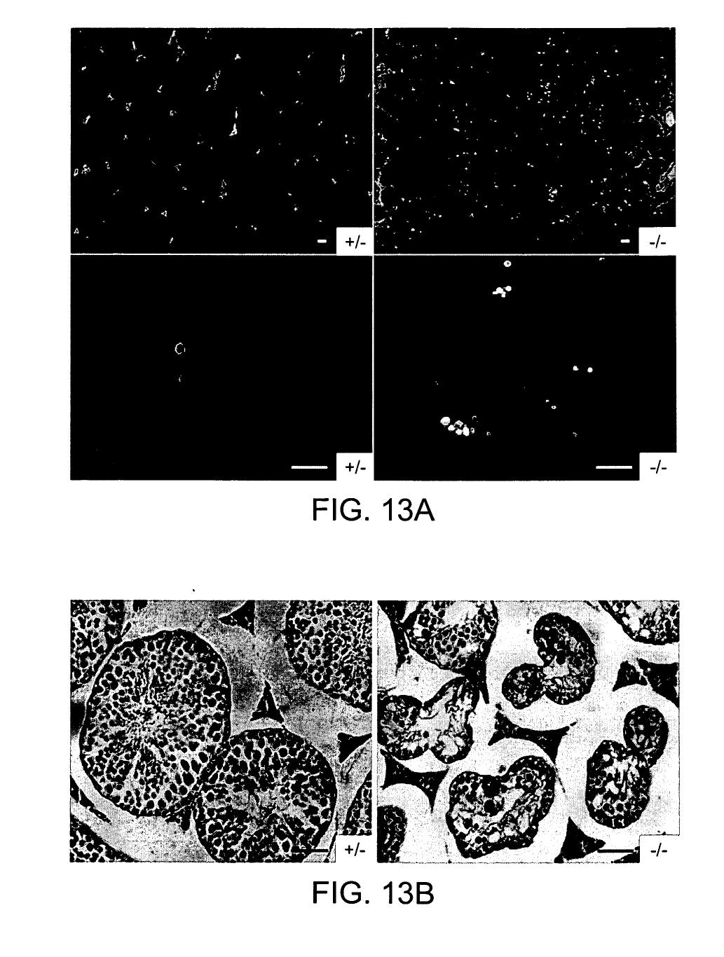

FIG. 13: Apoptic profile of adult testis in ANT-4 deficient mice The results of TUNEL labeling and caspase-3- staining to analyze the apoptotic profile of adult (6 wks) ANT4^{-/-} testis.

### DETAILED DESCRIPTION OF THE INVENTION

### 1. Definitions

Before further description of the present invention, and in order that the invention may be more readily understood, certain terms are first defined and collected here for convenience.

The term “administration” or “administering” includes routes of introducing the compound of the invention(s) to a subject to perform their intended function. Examples of routes of administration that may be used include injection (subcutaneous, intravenous, parenterally, intraperitoneally, intrathecal), oral, inhalation, rectal and transdermal. The pharmaceutical preparations may be given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the compound of the invention can be coated with or disposed in a selected material to protect it from natural conditions which may detrimentally affect its ability to perform its intended function. The compound of the invention can be administered alone, or in conjunction with either another agent as described above or with a pharmaceutically-acceptable carrier, or both. The compound of the invention can be administered prior to the administration of the other agent, simultaneously with the agent, or after the administration of the agent. Furthermore, the compound of the invention can also be administered in a proform which is converted into its active metabolite, or more active metabolite in vivo.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer, and still more preferably 4 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and sentences is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and still more preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiment, the term “lower alkyl” includes a straight chain alkyl having 4 or fewer carbon atoms in its backbone, e.g., C₁-C₄ alkyl.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonyl amino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The language “biological activities” of a compound of the invention includes all activities elicited by compound of the inventions in a responsive cell or subject. It includes genomic and non-genomic activities elicited by these compounds.

“Biological composition” or “biological sample” refers to a composition containing or derived from cells or biopolymers. Cell-containing compositions include, for example, mammalian blood, red cell concentrates, platelet concentrates, leukocyte concentrates, blood cell proteins, blood plasma, platelet-rich plasma, a plasma concentrate, a precipitate from any fractionation of the plasma, a supematant from any fractionation of the plasma, blood plasma protein fractions, purified or partially purified blood proteins or other components, serum, semen, mammalian colostrum, milk, saliva, placental extracts, a cryoprecipitate, a cryosupematant, a cell lysate, mammalian cell culture or culture medium, products of fermentation, ascites fluid, proteins induced in blood cells, and products produced in cell culture by normal or transformed cells (e.g., via recombinant DNA or monoclonal antibody technology). Biological compositions can be cell-free. In a preferred embodiment, a suitable biological composition or biological sample is a red blood cell suspension. In some embodiments, the blood cell suspension includes mammalian blood cells. Preferably, the blood cells are obtained from a human, a non-human primate, a dog, a cat, a horse, a cow, a goat, a sheep or a pig. In preferred embodiments, the blood cell suspension includes red blood cells and/or platelets and/or leukocytes and/or bone marrow cells. In some embodiments, the sample includes a marker (e.g., genetic material, mutant form, chemical or biological tag or identifier) suitable for following disorder or disease progression.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “effective amount” includes an amount effective, at dosages and for periods of time necessary, to achieve the desired result, e.g., sufficient to treat a cell proliferation related disorder or disease or an associated condition. An effective amount of compound of the invention may vary according to factors such as the disease state, age, and weight of the subject, and the ability of the compound of the invention to elicit a desired response in the subject. Dosage regimens may be adjusted to provide the optimum therapeutic response. An effective amount is also one in which any toxic or detrimental effects (e.g., side effects) of the compound of the invention are outweighed by the therapeutically beneficial effects.

A therapeutically effective amount of compound of the invention (i.e., an effective dosage) may range from about 0.001 to 30 mg/kg body weight, or from about 0.01 to 10 mg/kg body weight, or from about 0.05 to 5 mg/kg body weight, or from about 0.1 to 1 mg/kg, 0.2 to 0.9 mg/kg, 0.3 to 0.8 mg/kg, 0.4 to 0.7 mg/kg, or 0.5 to 0.6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound of the invention can include a single treatment or, preferably, can include a series of treatments. In one example, a subject is treated with a compound of the invention in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a compound of the invention used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The term “haloalkyl” is intended to include alkyl groups as defined above that are mono-, di- or polysubstituted by halogen, e.g., fluoromethyl and trifluoromethyl.

The term “halogen” designates —F, —Cl, —Br or —I.

The term “hydroxyl” means —OH.

The term “heteroatom” as used herein means an atom of any element other than carbon or hydrogen. Preferred heteroatoms are nitrogen, oxygen, sulfur and phosphorus.

The term “homeostasis” is art-recognized to mean maintenance of static, or constant, conditions in an internal environment.

The language “improved biological properties” refers to any activity inherent in a compound of the invention that enhances its effectiveness in vivo. In a preferred embodiment, this term refers to any qualitative or quantitative improved therapeutic property of a compound of the invention, such as reduced toxicity.

The term “optionally substituted” is intended to encompass groups that are unsubstituted or are substituted by other than hydrogen at one or more available positions, typically 1, 2, 3, 4 or 5 positions, by one or more suitable groups (which may be the same or different). Such optional substituents include, for example, hydroxy, halogen, cyano, nitro, C₁-C₈alkyl, C₃-C₈cycloalkyl, C₂-C₈ alkenyl, C₂-C₈alkynyl, C₁-C₈alkoxy, C₂-C₈alkyl ether, C₃-C₈alkanone, Cᵢ-C₈alkylthio, amino, mono- or di-(C₁-C₈alkyl)amino, haloC₁-C₈alkyl, C₁-C₈alkoxy, C₁-C_{g}alkanoyl, C₂-C₈alkanoyloxy, C₁-C₈alkoxycarbonyl, —COOH, —CONH₂, mono- or di-(C₁-C₈alkyl)aminocarbonyl, —SO₂NH₂, and/or mono or di(C₁-C₈alkyl)sulfonamido, as well as carbocyclic and heterocyclic groups. Optional substitution is also indicated by the phrase “substituted with from 0 to X substituents,” where X is the maximum number of possible substituents. Certain optionally substituted groups are substituted with from 0 to 2, 3 or 4 independently selected substituents (i.e., are unsubstituted or substituted with up to the recited maximum number of substitutents).

The term “isomers” or “stereoisomers” refers to compounds which have identical chemical constitution, but differ with regard to the arrangement of the atoms or groups in space.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and infrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “prodrug” includes compounds with moieties that can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “cell proliferation related disorder or disease” refers to diseases or conditions related to uncontrolled or disregulated cell growth or proliferation. Exemplary cell proliferation related disorders or diseases include, but are not limited to cancer, atherosclerosis, amyloidosis, polycythemia vera, and restenosis. Cancers include cancer of the colon, breast, bone, brain and others (e.g., osteosarcoma, neuroblastoma, colon adenocarcinoma), cardiac cancer (e.g., sarcoma, myxoma, rhabdomyoma, fibroma, lipoma and teratoma); lung cancer (e.g., bronchogenic carcinoma, alveolar carcinoma, bronchial adenoma, sarcoma, lymphoma, chondromatous hamartoma, mesothelioma); various gastrointestinal cancer (e.g., cancers of esophagus, stomach, pancreas, small bowel, and large bowel); genitourinary tract cancer (e.g., kidney, bladder and urethra, prostate, testis; liver cancer (e.g., hepatoma, cholangiocarcinoma, hepatoblastoma, angiosarcoma, hepatocellular adenoma, hemangioma); bone cancer (e.g., osteogenic sarcoma, fibrosarcoma, malignant fibrous histiocytoma, chondrosarcoma, Ewing's sarcoma, malignant lymphoma, multiple myeloma, malignant giant cell tumor chordoma, osteochronfroma, benign chondroma, chondroblastoma, chondromyxofibroma, osteoid osteoma and giant cell tumors); cancers of the nervous system (e.g., of the skull, meninges, brain, and spinal cord); gynecological cancers (e.g., uterus, cervix, ovaries, vulva, vagina); hematologic cancer (e.g., cancers relating to blood, Hodgkin's disease, non-Hodgkin's lymphoma); skin cancer (e.g., malignant melanoma, basal cell carcinoma, squamous cell carcinoma, Karposi's sarcoma, moles dysplastic nevi, lipoma, angioma, dermatofibroma, keloids, psoriasis); and cancers of the adrenal glands (e.g., neuroblastoma). In one aspect, the cancer is a type III germ cell tumor or anti-seminoma cancer.

As used herein, “obtaining a biological sample from a subject,” includes obtaining a sample for use in the methods described herein. A biological sample is described above.

The language “a prophylactically effective amount” of a compound refers to an amount of a compound of the invention of the formula (I) or otherwise described herein which is effective, upon single or multiple dose administration to the patient, in preventing or treating a cell proliferation related disorder or disease, or inhibiting spermatogenesis.

The language “reduced toxicity” is intended to include a reduction in any undesired side effect elicited by a compound of the invention when administered in vivo.

The language “selective ANT4 inhibitor” refers to a compounds capable of inhibiting the activity of ANT4 while not substantially inhibiting the activity of at least one of ANT1, ANT2, and ANT3 at the same compound concentration. For example, a selective ANT4 inhibitor may have ANT4 inhibitory activity 2, 5, 10, 20, 50, 100, 500, or 1000-fold more potent than its ANT1, ANT2, or ANT3 inhibitory activity. In certain embodiments, the ANT4 inhibitory activity of a selective ANT4 inhibitor is at least 5-fold, 10-fold, or 20-fold more potent than the ANT1 inhibitory activity of the compound.

The term “sulfhydryl” or “thiol” means —SH.

The term “subject” includes organisms which are capable of suffering from a cell proliferation related disorder or disease, desire or are in need of spermatogenesis inhibition (e.g., male contraception), or who could otherwise benefit from the administration of a compound of the invention of the invention, such as human and non-human animals. Preferred human animals include human patients suffering from or prone to suffering from a cell proliferative disease or disorder, as described herein. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc. “Susceptible to a cell proliferative disease or disorder” is meant to include subjects at risk of developing a cell proliferative disease or disorder, including cancer.

The phrases “systemic administration,” “administered systemically”, “peripheral administration” and “administered peripherally” as used herein mean the administration of a compound of the invention(s), drug or other material, such that it enters the patient's system and, thus, is subject to metabolism and other like processes, for example, subcutaneous administration.

The language “therapeutically effective amount” of a compound of the invention of the invention refers to an amount of an agent which is effective, upon single or multiple dose administration to the patient, in treating or preventing a cell proliferative disease or disorder or an associated condition or symptom, or in prolonging the survivability of the patient with such condition beyond that expected in the absence of such treatment.

With respect to the nomenclature of a chiral center, terms “d” and “l” configuration are as defined by the IUPAC Recommendations. As to the use of the terms, diastereomer, racemate, epimer and enantiomer will be used in their normal context to describe the stereochemistry of preparations.

### 2. Compounds of the Invention

In one aspect, the invention provides a compound capable of inhibiting ANT4 activity. In certain embodiments, the compound is capable of inhibiting ANT4 activity selectively, e.g., without concomitant inhibition of other ANTs, including ANT1, ANT2, or ANT3.

In certain embodiments, the ANT4 inhibitor compound can be represented by compounds of Table 1, or a pharmaceutically acceptable salt or prodrug thereof

In general, a compound of the invention will be selected such that the compound is capable of binding to a binding site or pocket of ANT4 that is defined (at least in part) by structure coordinates of one or more of ANT4 amino acid residues 62-63 and 257-261 of mouse ANT4. Moreover, in certain embodiments, a compound has one or more of the following properties: (1) not more than 5 hydrogen bond donors; (2) not more than 10 hydrogen bond acceptors; (3) a molecular weight of 1000 or less, 800 or less, 600 or less, 500 or less; and (4) a partition coefficient log P of less than 5.

Compounds according to the invention can generally be made according to techniques known in the field (see, e.g., Comprehensive Organic Synthesis, Trost, B. M. and Fleming, I. eds., Pergamon Press, Oxford; and references cited therein). Furthermore, compounds of the invention can be purified, separated, or isolated, e.g., by crystallization, chromatographic separation (e.g., by liquid chromatography), or by other methods known in the art.

Naturally occurring or synthetic isomers can be separated in several ways known in the art. Methods for separating a racemic mixture of two enantiomers include chromatography using a chiral stationary phase (see, e.g., “Chiral Liquid Chromatography,” W.J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

### 3. Uses of the Compounds Of The Invention

As described herein below, it has now been found that the compounds of the invention and analogs can treat and prevent cell proliferative diseases and disorders, including cancer.

Thus, in one aspect, the invention provides a method of treating a subject suffering from or susceptible to a cell proliferation related disorder or disease, the method comprising administering to a subject in need thereof a therapeutically effective amount of a compound delineated herein (e.g., an adenine nucleotide translocase 4 (ANT4) inhibitor), to thereby treat the subject suffering from or susceptible to a cell proliferation related disorder or disease. The ANT4 inhibitor acts to impair growth and/or induce death of abnormal cells. In one aspect, the compound is a selective ANT4 inhibitor compound (e.g., as demonstrated by protocols know in the art, including those delineated hereon). In one embodiment, the compound is capable of binding to or interacting with a binding site or pocket defined (at least in part) by structure coordinates of one or more ANT4 amino acid residues selected from amino acid residues 62-63 and 257-261 of mouse ANT4. In certain embodiments, the compound is a compound disclosed herein, e.g., a compound of Table 1. In certain embodiments, the subject is a mammal, e.g., a primate, e.g., a human.

In another aspect, the invention provides a method of treating a subject suffering from or susceptible to a cell proliferation related disorder or disease, the method comprising inhibiting the activity of adenine nucleotide translocase 4 (ANT4) in the subject such that the subject is treated.

In one aspect, the invention provides a method of inhibiting the growth of a cell, the method comprising inhibiting the activity of adenine nucleotide translocase 4 (ANT4) in the cell such that growth of the cell is inhibited. In one embodiment, the compound is capable of binding to or interacting with a binding site or pocket defined (at least in part) by structure coordinates of one or more ANT4 amino acid residues 62-63 and 257-261 of mouse ANT4. In another embodiment, the ANT4 inhibitor compound is demonstrated to bind to ANT4 at an amino acid sequence comprising ANT4 amino acid residues 62-63 and 257-261 of mouse ANT4. In certain embodiments, the compound is a compound disclosed herein, e.g., a compound of Table 1.

In one aspect, the invention provides a method of killing a cell, the method comprising inhibiting the activity of adenine nucleotide translocase 4 (ANT4) in the cell such that the cell is killed. In one aspect the cell is a cancer cell.

In another aspect, the invention provides a method for inhibiting activity or expression of ANT4 (e.g., in vitro, in vivo) in a cell or a subject, the method comprising contacting the cell or subject with an effective amount of a compound capable of inhibiting activity of ANT4, such that activity of ANT4 is inhibited. In another aspect, the method is any of those herein wherein the activity of adenine nucleotide translocase 4 (ANT4) in the cell is inhibited by contacting the cell with a selective inhibitor of ANT4.

In another aspect, the invention provides a method for inhibiting sperm motility in a cell or subject, the method comprising contacting the cell or subject with an effective amount of a compound capable of inhibiting activity of ANT4, such that activity of ANT4 is inhibited. In another aspect, the sperm motility in the subject returns upon discontinuation of administration of the ANT4 inhibitor compound.

In another aspect, the invention provides a method for inducing contraception in a male subject, the method comprising administering to the subject an effective amount of a compound capable of inhibiting activity of ANT4, such that activity of ANT4 is inhibited. In another aspect, the contraception effect is for a limited time. In another aspect, the contraception effect is reversible.

In another aspect, the invention provides a method for reducing or eliminating male meiotic spermatocytes (without damaging other cell types) in a subject, the method comprising administering to the subject a compound that inhibits adenine nucleotide translocase 4 (ANT4), such that activity of ANT4 is inhibited.

In another aspect, the invention provides a method for controlling (e.g., reducing, inhibiting proliferation, inhibiting reproduction) animal populations by inducing contraception in a male subject (e.g., a pest mammal, rodent, mouse, rat, rabbit) comprising administering to the subject a compound that inhibits adenine nucleotide translocase 4 (ANT4).

The present methods can be performed on cells in culture, e.g. in vitro or ex vivo, or on cells present in an animal subject, e.g., in vivo. Compounds of the inventions can be initially tested in vitro using primary cultures of cells.

The present methods can be performed on cells in culture, e.g. in vitro or ex vivo, or on cells present in an animal subject, e.g., in vivo. Compounds of the invention can be initially tested in vitro using cells from the respiratory tract from embryonic rodent pups (See e.g. U.S. Pat. No. 5,179,109-fetal rat tissue culture), or other mammalian (See e.g. U.S. Pat. No. 5,089,517-fetal mouse tissue culture) or non-mammalian animal models.

Alternatively, the effects of a compound of the invention can be characterized in vivo using animals models.

In one aspect, the methods herein modulate (e.g, inhibit, kill, induce apopotosis, reduce proliferation) cells that are cancer cells.

In one aspect, the methods herein modulate (e.g, inhibit, kill, induce apopotosis, reduce proliferation, inhibit motility) sperm cells.

In another aspect, the methods herein are those wherein the modulation (of ANT4) occurs with reduced or lessened side effects (e.g., undesired effects due to modulation of ANT1, ANT2, ANT3, or some other biological target) than seen with less selective compounds than those delineated herein.

In certain embodiments, the methods of the invention include administering to a subject a therapeutically effective amount of a compound of the invention in combination with another pharmaceutically active compound. In a further embodiment, the additional therapeutic agent is an anti-cancer agent, chemotherapeutic agent, an anti-angiogenesis agent, cytotoxic agent, or an anti-proliferation agent. Examples of such chemotherapeutic agents include but are not limited to daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine (CA), 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, vincristine, vinblastine, etoposide, trimetrexate, teniposide, cisplatin and diethylstilbestrol (DES). See, generally, The Merck Manual of Diagnosis and Therapy, 15th Ed., pp. 1206-1228, Berkow et al., eds., Rahway, N.J., 1987). The compound of the invention and the pharmaceutically active compound may be administered to the subject in the same pharmaceutical composition or in different pharmaceutical compositions (at the same time or at different times).

Determination of a therapeutically effective amount or a prophylactically effective amount of the compound of the invention, can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician; the severity of the condition being treated and the particular compound being employed. In determining the therapeutically effective amount or dose, and the prophylactically effective amount or dose, a number of factors are considered by the attending clinician, including, but not limited to: the specific cell proliferative disease or disorder involved; pharmacodynamic characteristics of the particular agent and its mode and route of administration; the desired time course of treatment; the species of mammal; its size, age, and general health; the degree of or involvement or the severity of the disease; the response of the individual patient; the particular compound administered; the mode of administration; the bioavailability characteristics of the preparation administered; the dose regimen selected; the kind of concurrent treatment (i.e., the interaction of the compound of the invention with other co-administered therapeutics); and other relevant circumstances.

Treatment can be initiated with smaller dosages, which are less than the optimum dose of the compound. Thereafter, the dosage may be increased by small increments until the optimum effect under the circumstances is reached. For convenience, the total daily dosage may be divided and administered in portions during the day if desired. A therapeutically effective amount and a prophylactically effective amount of a compound of the invention of the invention is expected to vary from about 0.1 milligram per kilogram of body weight per day (mg/kg/day) to about 100 mg/kg/day.

Compounds determined to be effective for the prevention or treatment of cell proliferative diseases or disorders in animals, e.g., dogs, chickens, and rodents, may also be useful in treatment of similar conditions in humans. Those skilled in the art of treatment in humans will know, based upon the data obtained in animal studies, the dosage and route of administration of the compound to humans. In general, the dosage and route of administration in humans is expected to be similar to that in animals.

In one aspect, the methods herein are those wherein the subject is identified as in need of treatment by administration of a compound herein (e.g., an ANT4 inhibitor compound). In one aspect, the methods herein are those wherein the subject is identified as in need of treatment by administration of a compound herein (e.g., an ANT4 inhibitor compound) that is demonstrated to bind to ANT4 at a site comprising amino acid residues 62-63 and 257-261 of mouse ANT4. The identification of those patients who are in need of prophylactic treatment for cell proliferative diseases or disorders is well within the ability and knowledge of one skilled in the art. Certain of the methods for identification of patients which are at risk of developing cell proliferative diseases or disorders which can be treated by the subject methods are appreciated in the medical arts, such as family history, the presence of other risk factors associated with the development of that disease state in the subject patient, and the like. A clinician skilled in the art can readily identify such candidate patients, by the use of, for example, clinical tests, physical examination and medical/family/travel history.

A method of assessing the efficacy of a disease treatment in a subject includes determining the physical condition of the subject (e.g., blood pressure, degree or extent of disorder progression, obtaining a biological sample, and the like) and then administering a therapeutically effective amount of an ANT4 inhibitor compound of the invention to the subject. After a appropriate period of time after the administration of the compound, e.g., 2 hours, 4 hours, 8 hours, 12 hours, or 72 hours, or one week, the physical condition of the subject is determined again. The modulation of the cell proliferative disease or disorder indicates efficacy of an treatment. The physical condition of the subject may be determined periodically throughout treatment. For example, the physical condition of the subject may be checked every few hours, days or weeks to assess the further efficacy of the treatment. The method described may be used to screen or select patients that may benefit from treatment with an ANT4 inhibitor.

In another aspect, the invention provides a method for identifying a compound that modulates (e.g., inhibits) the activity of ANT4, the method comprising using the atomic coordinates of one or more of ANT4 amino acid residues 62-63 and 257-261 of mouse ANT4 to generate a three-dimensional structure of a molecule comprising an ANT4 binding pocket, and employing the three-dimensional structure to identify a compound that modulates the activity of ANT4.

In another aspect, a compound of the invention is packaged in a therapeutically effective amount with a pharmaceutically acceptable carrier or diluent. The composition may be formulated for treating a subject suffering from or susceptible to a cell proliferation related disorder or disease, and packaged with instructions to treat a subject suffering from or susceptible to such a disease or condition. In aspects, the method of packaging comprises combining the compound or compositions delineated herein in a pharmaceutically acceptable packaging material, In other aspects the method of packaging comprises inserting, printing or attaching instructions (e.g., insert, printing on package, label) in or on the packaging.

In another aspect, the invention provides a packaged composition including a therapeutically effective amount of an ANT4 inhibitor compound and a pharmaceutically acceptable carrier or diluent. The composition may be formulated for treating a subject suffering from or susceptible to a cell proliferation related disorder or disease, and packaged with instructions to treat a subject suffering from or susceptible to a cell proliferation related disorder or disease.

In one aspect, the invention provides a kit for treating a cell proliferation related disorder or disease in a subject is provided and includes a compound disclosed herein, e.g., a compound of Table 1, or a pharmaceutically acceptable ester, salt, and prodrug thereof, and instructions for use. In further aspects, the invention provides kits for treating a cell proliferation related disorder or disease, assessing the efficacy of an anti-cell-proliferative treatment in a subject using an ANT4 inhibitor, monitoring the progress of a subject being treated with an ANT4 inhibitor, selecting a subject with or susceptible to a cell proliferation related disorder or disease, and/or treating a subject suffering from or susceptible to a cell proliferation related disorder or disease. In certain embodiments, the invention provides: a kit for treating a cell proliferation related disorder or disease in a subject, the kit comprising a compound capable of inhibiting activity (or expression) of ANT4, or pharmaceutically acceptable esters, salts, and prodrugs thereof, and instructions for use; in certain embodiments, the compound is represented any of the structures of Table 1, or a pharmaceutically acceptable salt thereof.

### 4. Pharmaceutical Compositions

The invention also provides a pharmaceutical composition, comprising an effective amount of a compound of the invention, e.g., a compound of Table 1, or otherwise described herein and a pharmaceutically acceptable carrier. In a further embodiment, the effective amount is effective to treat a cell proliferation related disorder or disease, as described previously.

In an embodiment, the compound of the invention is administered to the subject using a pharmaceutically-acceptable formulation, e.g., a pharmaceutically-acceptable formulation that provides sustained delivery of the compound of the invention to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier is “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (1₃) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a compound of the invention(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, more preferably from about 10 percent to about 30 percent.

Methods of preparing these compositions include the step of bringing into association a compound of the invention(s) with the carrier and, optionally, one or more accessory ingredients. In general, the formulations are prepared by uniformly and intimately bringing into association a compound of the invention with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compound of the invention(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active compound of the invention(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more compound of the invention(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a compound of the invention(s) include powders, sprays, ointments, pastes, creams, lotions, gels, solutions, patches and inhalants. The active compound of the invention(s) may be mixed under sterile conditions with a pharmaceutically-acceptable carrier, and with any preservatives, buffers, or propellants which may be required.

The ointments, pastes, creams and gels may contain, in addition to compound of the invention(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of the invention(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compound of the invention(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a compound of the invention(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of the invention.

Pharmaceutical compositions of the invention suitable for parenteral administration comprise one or more compound of the invention(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of compound of the invention(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compound of the invention(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the compound of the invention(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. An exemplary dose range is from 0.01 to 10 mg per day.

A preferred dose of the compound of the invention for the present invention is the maximum that a patient can tolerate and not develop serious or unacceptable side effects. In certain embodiments, the compound of the present invention is administered at a concentration of about 10 micrograms to about 100 mg per kilogram of body weight per day, about 0.1-about 10 mg/kg or about 1.0 mg-about 10 mg/kg of body weight per day. Ranges intermediate to the above-recited values are also intended to be part of the invention.

5. Screening Methods and Systems

In another aspect, the invention provides a method for identifying a compound that inhibits ANT4 activity (e.g., a chemotherapeutic agent, male contraceptive agent, sperm motility inhibitor), the method comprising obtaining a structure of ANT4 or obtaining information relating to the structure of ANT4, and modeling a test compound into or on the crystal structure coordinates to determine whether the compound inhibits ANT4. In certain embodiments, the step of modeling comprises modeling or determining the ability of the compound to bind to or associate with a binding pocket defined by structure coordinates of one or more ANT4 amino acid residues 62-63 and 257-261 of mouse ANT4.

Yet another aspect of the invention is a method for identifying a compound that modulates the activity of ANT4, the method comprising using the atomic coordinates of one or more ANT4 amino acid residues 62-63 and 257-261 of mouse ANT4 to generate a three-dimensional structure of a molecule comprising an ANT4 binding site or binding pocket, and employing the three-dimensional structure to identify a compound that modulates (e.g., binds, inhibits) the activity of ANT4.

In another aspect, the invention relates to a three-dimensional structure of ANT4. The invention provides key structural features of ANT4.

In another aspect, the invention provides a machine readable storage medium which comprises the structural coordinates of either one or both of the binding pockets identified herein, or similarly shaped, homologous binding pockets. Such storage media encoded with these data are capable of displaying a three-dimensional graphical representation of a molecule or molecular complex which comprises such binding pockets on a computer screen or similar viewing device. Thus, in one embodiment, the invention provides a machine readable storage medium which comprises the structural coordinates of a binding pocket defined (at least in part) by structure coordinates of one or more of ANT4 amino acid residues 62-63 and 257-261 of mouse ANT4, or a homologous binding site or binding pocket.

In another aspect, the invention provides a computer for producing a three-dimensional representation of a molecule or molecular complex, wherein said molecule or molecular complex comprises a binding pocket defined by structural coordinates of a binding pocket defined (at least in part) by structure coordinates of one or more of ANT4 amino acid residues 62-63 and 257-261 of mouse ANT4, or a homologous binding site or pocket; or b) a three-dimensional representation of a homologue of said molecule or molecular complex, wherein said homologue comprises a binding pocket that has a root mean square deviation from the backbone atoms of said amino acids of not more than about 2.0 angstroms. The computer includes (i) a machine-readable data storage medium comprising a data storage material encoded with machine-readable data, wherein said data comprises the structural coordinates of a binding pocket defined (at least in part) by structure coordinates of one or more of ANT4 amino acid residues 62-63 and 257-261 of mouse ANT4, or a homologous binding site pocket; (ii) a working memory for storing instructions for processing said machine-readable data; (iii) a central-processing unit coupled to said working memory and to said machine-readable data storage medium for processing said machine readable data into said three-dimensional representation; and (iv) a display coupled to said central-processing unit for displaying said three-dimensional representation.

Thus, the computer produces a three-dimensional graphical structure of a molecule or a molecular complex which comprises a binding site or pocket.

In another embodiment, the invention provides a computer for producing a three-dimensional representation of a molecule or molecular complex defined by structure coordinates of all or some of the ANT4 amino acids, or a three-dimensional representation of a homologue of said molecule or molecular complex, wherein said homologue comprises a binding pocket that has a root mean square deviation from the backbone atoms of said amino acids of not more than 2.0 (more preferably not more than 1.5) angstroms.

In exemplary embodiments, the computer or computer system can include components which are conventional in the art, e.g., as disclosed in U.S. Pat. No. 5,978,740 and/or 6,183,121 (incorporated herein by reference). For example, a computer system can includes a computer comprising a central processing unit (“CPU”), a working memory (which may be, e.g., RAM (random-access memory) or “core” memory), a mass storage memory (such as one or more disk drives or CD-ROM drives), one or more cathode-ray tube (CRT) or liquid crystal display (LCD) display terminals, one or more keyboards, one or more input lines, and one or more output lines, all of which are interconnected by a conventional system bus.

Machine-readable data of this invention may be inputted to the computer via the use of a modem or modems connected by a data line. Alternatively or additionally, the input hardware may include CD-ROM drives, disk drives or flash memory. In conjunction with a display terminal, a keyboard may also be used as an input device.

Output hardware coupled to the computer by output lines may similarly be implemented by conventional devices. By way of example, output hardware may include a CRT or LCD display terminal for displaying a graphical representation of a binding pocket of this invention using a program such as QUANTA or PYMOL. Output hardware might also include a printer, or a disk drive to store system output for later use.

In operation, the CPU coordinates the use of the various input and output devices, coordinates data accesses from the mass storage and accesses to and from working memory, and determines the sequence of data processing steps. A number of programs may be used to process the machine-readable data of this invention, including commercially-available software.

A magnetic storage medium for storing machine-readable data according to the invention can be conventional. A magnetic data storage medium can be encoded with a machine-readable data that can be carried out by a system such as the computer system described above. The medium can be a conventional floppy diskette or hard disk, having a suitable substrate which may be conventional, and a suitable coating, which may also be conventional, on one or both sides, containing magnetic domains whose polarity or orientation can be altered magnetically. The medium may also have an opening for receiving the spindle of a disk drive or other data storage device.

The magnetic domains of the medium are polarized or oriented so as to encode in manner which may be conventional, machine readable data such as that described herein, for execution by a system such as the computer system described herein.

An optically-readable data storage medium also can be encoded with machine-readable data, or a set of instructions, which can be carried out by a computer system. The medium can be a conventional compact disk read only memory (CD-ROM) or a rewritable medium such as a magneto-optical disk which is optically readable and magneto-optically writable.

In the case of CD-ROM, as is well known, a disk coating is reflective and is impressed with a plurality of pits to encode the machine-readable data. The arrangement of pits is read by reflecting laser light off the surface of the coating. A protective coating, which preferably is substantially transparent, is provided on top of the reflective coating.

In the case of a magneto-optical disk, as is well known, a data-recording coating has no pits, but has a plurality of magnetic domains whose polarity or orientation can be changed magnetically when heated above a certain temperature, as by a laser. The orientation of the domains can be read by measuring the polarization of laser light reflected from the coating. The arrangement of the domains encodes the data as described above.

Structure data, when used in conjunction with a computer programmed with software to translate those coordinates into the 3-dimensional structure of a molecule or molecular complex comprising a binding pocket, may be used for a variety of purposes, such as drug discovery. The structure can be an ANT structure having the amino acid sequence of any delineated herein, or fragment thereof.

For example, the structure encoded by the data may be computationally evaluated for its ability to associate with chemical entities. Chemical entities that associate with a binding site or pocket of ANT4 as disclosed herein may inhibit ANT4 activity, and are potential drug candidates. Alternatively, the structure encoded by the data may be displayed in a graphical three-dimensional representation on a computer screen. This allows visual inspection of the structure, as well as visual inspection of the structure's association with chemical entities.

Thus, according to another embodiment, the invention relates to a method for evaluating the potential of a chemical entity to associate with a) a molecule or molecular complex comprising a binding pocket defined, at least in part, by structure coordinates of one or more ANT4 amino acid residues 62-63 and 257-261 of mouse ANT4, as described herein, or b) a homologue of said molecule or molecular complex, wherein said homologue comprises a binding pocket that has a root mean square deviation from the backbone atoms of said amino acids of not more than 2.0 (more preferably 1.5) angstroms.

This method comprises the steps of:

i) employing computational means to perform a fitting operation between the chemical entity and a binding pocket of the molecule or molecular complex; and

ii) analyzing the results of the fitting operation to quantify the association between the chemical entity and the binding pocket.

The term “chemical entity”, as used herein, refers to chemical compounds, complexes of at least two chemical compounds, and fragments of such compounds or complexes.

In certain embodiments, the method evaluates the potential of a chemical entity to associate with a molecule or molecular complex defined by structure coordinates of all or some of the amino acids of ANT4, as described herein, or a homologue of said molecule or molecular complex having a root mean square deviation from the backbone atoms of said amino acids of not more than 2.0 (more preferably not more than 1.5) angstroms.

In a further embodiment, the structural coordinates one of the binding pockets described herein can be utilized in a method for identifying a potential agonist or antagonist of a molecule comprising an ANT4 binding site or pocket. This method comprises the steps of:

a) using the atomic coordinates of ANT4 amino acid residues 62-63 and 257-261 of mouse ANT4, as described herein, with a root mean square deviation from the backbone atoms of said amino acids of not more than about 2.0 (more preferably not more than 1.5) angstroms, to generate a three-dimensional structure of molecule comprising an ANT4 binding site or pocket;

b) employing the three-dimensional structure to design or select the potential agonist or antagonist. The method further includes the optional steps of c) synthesizing the agonist or antagonist; and d) contacting the agonist or antagonist with the molecule to determine the ability of the potential agonist or antagonist to interact with the molecule.

In another aspect, the invention is a method of identifying a compound useful for inhibiting ANT4 activity comprising the steps of:
1. Obtaining the crystal structure of human ANT4, whereby a three-dimensional structure of ANT4 will be generated;
2. Determination of ANT4 binding regions or pockets by mapping the atomic coordinates of selected amino acid residues 62-63 and 257-261 of mouse ANT4, to provide an ANT4 modeling system, then providing a machine readable storage medium which comprises the three-dimensional ANT4 structure and binding pocket(s) defined (at least in part) by structure coordinates of one or more of ANT4 amino acid residues 62-63 and 257-261 of mouse ANT4;
3. Employing the ANT4 modeling system to identify test compounds that bind within the selected ANT4 binding pockets;
4. Determining the selectivity of compound inhibition, i.e. inhibiting ANT4 over ANT1 (or other ANT (e.g., ANT1, ANT2 or ANT3) or biological target), by measuring the ADP/ATP exchange for each ANT, following treatment with compound;
5. Identifying compounds that inhibit ANT4 to the extent of cell growth inhibition or cell death.

The present inventors' elucidation of heretofore unknown binding sites or pockets in the structure of ANT4 provides the necessary information for designing new chemical entities and compounds that may interact with ANT4, in whole or in part, and may therefore modulate (e.g., inhibit or decrease) the activity of ANT4, preferably with selectivity relative to other ANTs.

The design of compounds that bind to ANT4 binding sites or pockets according to this invention generally involves consideration of several factors. First, the entity must be capable of physically and structurally associating with parts or all of an ANT4 binding site or pocket. Non-covalent molecular interactions important in this association include hydrogen bonding, van der Waals interactions, hydrophobic interactions and electrostatic interactions. Second, the entity must be able to assume a conformation that allows it to associate with an ANT4 binding site or pocket directly. Although certain portions of the entity will not directly participate in these associations, those portions of the entity may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of an entity comprising several chemical entities that directly interact with the binding pocket or homologues thereof.

The potential inhibitory or binding effect of a chemical entity on an ANT4 binding site or pocket may be analyzed prior to its actual synthesis and testing by the use of computer modeling techniques. If the theoretical structure of the given entity suggests insufficient interaction and association between it and the target binding pocket, testing of the entity is obviated. However, if computer modeling indicates a strong interaction, the molecule may then be synthesized and tested for its ability to bind to a binding pocket. This may be achieved, e.g., by testing the ability of the molecule to inhibit ANT4 activity, e.g., using assays described herein or known in the art. In this manner, synthesis of inoperative compounds may be avoided.

A potential inhibitor of an ANT4-related binding pocket may be computationally evaluated by means of a series of steps in which chemical entities or fragments are screened and selected for their ability to associate with the ANT4-related binding pockets.

One skilled in the art may use one of several methods to screen chemical entities or fragments for their ability to associate with an ANT4 binding site or pocket. This process may begin by visual inspection of, for example, an ANT4 binding site or pocket on the computer screen based on the structure coordinates described herein, or other coordinates which define a similar shape generated from the machine-readable storage medium. Selected fragments or chemical entities may then be positioned in a variety of orientations, or docked, within that binding pocket as defined supra. Docking may be accomplished using software such as Quanta and DOCK, followed by energy minimization and molecular dynamics with standard molecular mechanics force fields, such as CHARMM and AMBER.

Specialized computer programs (e.g., as known in the art and/or commercially available and/or as described herein) may also assist in the process of selecting fragments or chemical entities.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of the target binding pocket.

Instead of proceeding to build a compound capable of binding to a binding pocket in a step-wise fashion one fragment or chemical entity at a time as described above, inhibitory or other binding compounds may be designed as a whole or “de novo” using either an empty binding site or optionally including some portion(s) of a known inhibitor(s). There are many de novo ligand design methods known in the art, some of which are commercially available (e.g., LeapFrog, available from Tripos Associates, St. Louis, Mo.).

Other molecular modeling techniques may also be employed in accordance with this invention (see, e.g., N. C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology” 4, pp. 777-781 (1994)).

Once a compound has been designed or selected, the efficiency with which that entity may bind to a binding pocket may be tested and optimized by computational evaluation.

Specific computer software is available in the art to evaluate compound deformation energy and electrostatic interactions. Examples of programs designed for such uses include: AMBER; QUANTA/CHARMM (Accelrys, Inc., Madison, Wis.) and the like. These programs may be implemented, for instance, using a commercially-available graphics workstation. Other hardware systems and software packages will be known to those skilled in the art.

Another technique involves the in silico screening of virtual libraries of compounds, e.g., as described herein (see, e.g., the Examples herein below). Many thousands of compounds can be rapidly screened and the best virtual compounds can be selected for further screening (e.g., by synthesis and in vitro testing). Small molecule databases can be screened for chemical entities or compounds that can bind, in whole or in part, to an ANT4 binding site or pocket. In this screening, the quality of fit of such entities to the binding site may be judged either by shape complementarity or by estimated interaction energy.

Finally, additional computational techniques can be used for automated structure-based optimization with software packages such as RACHEL (Tripos, Inc.). RACHEL allows a database of fragments to be screened and evaluated (i.e., scored) as each fragment is considered as an extension of the lead compound. The lead compound can then be grown in silico at user defined sites and ranked again. This approach can provide a “filtered” library of derivatives likely to have an increased affinity for the target.

In the methods described herein, the additional steps of procuring (e.g., purchasing or obtaining from a commercial source or compound library, synthesizing, etc.) the compound and/or testing the compound in a laboratory protocol (e.g., assay, experiment, etc.) may also be included.

The invention also provides methods for designing, evaluating and identifying compounds which bind to the aforementioned binding site/pocket. Such compounds are potential inhibitors or modulators of ANT4 activity. Other embodiments of the invention are disclosed herein.

The invention is further illustrated by the following examples which should in no way be construed as being further limiting.

### EXAMPLES

### Materials and Methods

### Virtual Screening

The software package of DOCKv5.2 (Ewing et al. 2001) was used for in silico screening of ˜240,000 compounds available from the National Cancer Institute, Developmental Therapeutics Program. The structure coordinates and chemical information for each compound were processed either with accessory software from DOCK or with the ZINC server (Irwin and Shoichet 2005).

The grid-based scoring system was used for scoring with the non-bonded force field energy function implemented in DOCK. A standard 6-12 Lennard-Jones potential was used to evaluate van der Waals contacts. Spheres were generated by SPHGEN (Kuntz et al. 1982) and clusters were edited by hand to target specific sites on the molecular surface of ANT4.

### Example 1

### Compound Modeling/Screening

Approximately 300,000 compounds were virtually screened with DOCKv5.2 (Ewing et al. 2001) and ranked by energy score. This computer database was prepared with DOCK accessory software (SF2MOL2, UCSF) and Sybyl (Tripos, Inc.).

We first predicted the three-dimensional structure of ANT4 (SWISS-MODEL) based on the solved crystal structure of bovine Ant1. The docking site was selected based on its unique specificity to ANT2 and not the other ANTs, and on characteristics favorable for small molecule binding.

The atomic model of mouse Ant4 is shown superimposed on the bovine Ant1 (FIG. 1). Non-protein atoms are shown as spheres and represent potential sites for intervention with small molecules.

The site selected for molecular docking is at the prominent cleft in the mouse Ant4 structure (FIG. 2). The amino acid sequence surrounding the cleft (amino acids 62-63 and 257-261 of mouse Ant4) is conserved within Ant4, but differs from Ant1, 2 and 3 (FIG. 3). Therefore, the docking site is unique to Ant4, but not somatic Ants. Importantly, the selected docking site (mainly contained in the M3 region) is located at the ADP/ATP binding site (FIG. 4). Thus, we predict that the small compound interaction at the site would interfere with ADP/ATP exchange through Ant4.

We utilized DOCK6 to carry out dynamic molecular docking simulations. The coordinates for approximately 300,000 compounds (most of which are available through the NCl/DTP) were used as the ligand database for molecular docking utilizing the prominent cleft site selected in the mouse Ant4 structure shown in FIG. 2. Each small molecule was positioned in the selected site in 100 different orientations, and the best orientations and their scores (contact and electrostatic) were calculated. The atomic positions and chemical characteristics of residues in close proximity (within 5 Å) to the selected site were used to establish a scoring grid to evaluate potential interactions with small molecules. Two types of interactions were scored: van der Waals contact and electrostatic interactions. The scored compounds were ranked and the 300 highest-scoring compounds will be requested for functional evaluation.

Further optimization of lead compounds is performed using RACHEL (Real-time Automated Combinatorial Heuristic Enhancement of Lead) technology to obtain compounds having higher affinities/biological activities (Ho 1990, 1993, 1995, Dammkoehler 1989). Starting from the mouse Ant4 structure and the orientations of active lead compounds posed by DOCK, RACHEL will perform automated combinatorial optimization of lead compounds by systematically derivatizing user-defined sites on the ligand. These compounds will be conformationally searched within the active site, evaluated, and only those that bind tightly with the structural pocket will be retained. This new population of compounds is then processed to form the next generation of derivatives. Over time, a lead compound is iteratively refined into a set of high affinity structures.

The initial step of derivatization with RACHEL is defining the orientation of the lead compound in the selected structural pocket. Next, ligand atoms are selected as “anchor” positions and target atoms (in the structure of Ant4, in this case) are defined to dictate the direction of growth for the new compounds. Finally, chemical groups are added to the lead compound and the new molecules are evaluated for their potential interactions with the targeted structural pocket. The highest scoring compounds are then evaluated to determine the feasibility of synthesis by organic methods. A set of the highest scoring derivatives, based on the structure of the most active inhibitors, are synthesized for lead optimization.

### Example 2

### Sperm Motility Assaying of Compounds

Although glycolysis is adequate for normal sperm motility, ATP from oxidative phosphorylation will become essential for sperm motility when glucose is not available. Mouse sperm express the Ant4 protein (FIG. 5), but do not express detectable levels of either Ant1 or Ant2 (data not shown). Therefore, inhibition of Ant4 would disrupt transport of ATP produced in the mitochondria, thus reducing available ATP, particularly in non-glycolytic conditions. To this end, we will test the ability of the compounds to inhibit motility of mouse sperm in a medium containing either pyruvate alone (non-glycolytic medium) or pyruvate plus glucose (glycolytic medium). If the compounds inhibit Ant4, we expect that sperm motility will be affected more profoundly in non-glycolytic medium. Mouse sperm will be prepared as described below, resuspended in either glycolytic or non-glycolytic medium on 96 well-plates with each compound at 50 μM for 30 min, and subsequently be subjected to qualitative motility evaluation under an inverted light microscope. Those compounds inhibiting sperm motility preferentially in non-glycolytic medium will be subjected to further quantitative assays for sperm motility and dose response experiments. As a negative control, we will use a vehicle alone (DMSO, final concentration 0.05% in medium). As a positive control, we will use bongkrekic acid, a general Ant inhibitor which prevents active movement of adenine nucleotides across the inner mitochondrial membrane through all Ant family members including Ant4 (Hendersen 1970, Dolce 2005). Bongkrekic acid (50 μM) is known to make sperm almost completely immotile (Van Dop 1977).

In addition, in order to exclude the possibility that the compounds inhibit sperm motility by general cellular toxicity, we will also examine the effects of the compounds on growth and viability of the somatic cells which express Ant2 alone. The mouse NIH3T3 embryonic fibroblast cell line expresses Ant2, but no other Ants. Mouse ES cells similarly express predominantly Ant2. Although a very low level of Ant4 mRNA was detectable by RT-PCR (Brower 2007), both Northern and Western blot analysis failed to detect any significant levels of Ant4 RNA and protein in mouse ES cells.

### Sperm Preparation and Assays for Sperm Motility:

To begin, both cauda epididymes are removed from adult male mice (C57B6, 6-8 weeks of age), punctured with sharp forceps, and spermatozoa are squeezed out of the cauda region (Kusakabe 2004). Sperm are allowed to disperse for 10 min in 0.5 ml modified Krebs-Ringer bicarbonate (mKRB) solution (94.6 mM NaCl, 4.78 mM KCl, 1.71 mM CaCl₂, 1.19 mM KH₂PO₄, 1.19 mM MgSO₄, 25.07 mM NaHCO₃, 21.58 mM Sodium lactate, 0.5 mM Sodium pyruvate, 5.56 mM Glucose, 4.0 mg/ml BSA, 50 μg/ml Streptomycin sulfate, 75 μg/ml Potassium penicillin). The pH of the medium is previously equilibrated to 7.4 at 37° C. overnight in 5% CO₂ (Oh 1998). Motile sperm are collected by a swim-up procedure (Wu 2004). Briefly, swim-up is performed by gently adding a 1 ml overlay of mKRB to the 0.5 ml of isolated sperm. Tubes are placed at a 45 degree angle and incubated for 30 min at 37° C. in 5% CO₂. After incubation, 1.0 ml of the supernatant is collected, and samples are spun at 300 g for 10 min. The resultant pellet is resuspended in 0.25 ml volume of mKRB with or without glucose for analysis.

In the initial screening step, the effects of test compounds (50 μM final concentration) are assessed for motility inhibition. The experiments are performed in triplicate. Scoring is performed by two non-biased individuals. Motility is scored as follows. (+++) active motility equivalent to controls, (++) moderate reduction of motility, (+) severe reduction of motility, (−) nearly immotile.

Those compounds affecting sperm motility specifically in mKRB without glucose compared to sperm in the buffer containing glucose are further assessed in a second screening step. Epididymal sperm is isolated and prepared as described above. An aliquot of sample is added to Cell-Vu fixed coverslip disposable counting chamber (Erie Scientific Company, Portsmouth, N.H.) and analyzed using computer assisted sperm analysis (CASA) (Hamilton-Thorne IVOS V12.2L; Hamilton Thome Research, located at Gamete Genetics Lab, M341, UF College of Medicine). Parameters are assessed using a 60 Hz dark field video source with a 37° C. stage temperature. At least 300 motile sperm and five fields are assessed by CASA for each treatment group. Parameters assessed in this study include: motility (%), curvilinear velocity (VCL, um/sec), straight-line velocity (VSL, um/sec), average path velocity (VAP, gm/sec), amplitude of lateral head displacement (ALH, μm), beat-cross frequency (BCF, Hz), linearity (LIN=VSL/VCL, %), and percentage of rapid sperm (VAP>25 gm/sec, %). The CASA motility parameters used include those recommended by the manufacturer for mouse sperm.

The 14 compounds that had the highest scores for potential ANT4 binding were selected and examined their effects on mouse sperm motility. The compounds were obtained from NCI Developmental Therapeutics Program.

One compound (#12, NSC-42233) inhibited sperm motility as shown in FIG. 6. Similar results were obtained with three independent experiments. A preferential inhibition of sperm motility in non-glycolytic conditions supports the idea that the compound may be a true Ant4 inhibitor. Further, the #12 compound did not inhibit the cell growth or viability of mouse ES cells or NIH3T3 cells up to 50 μM (data not shown) indicating that the effects on sperm motility is not likely due to non-specific general cell toxicity.

The structures of these compounds are shown below:

### TABLE 1

### Compound #12 (NSC-42233):

3-acetyl-6-methyl-heptane-2,4-dione

Conclusion: One out of the 14 small compounds selected for ANT4 binding showed sperm inhibition activity.

### Example 3

### Study of ANT Selectivity

The test compounds are tested for their ability to selectively inhibit ADP/ATP exchange through Ant4 over other Ants which are reconstituted on liposomes (FIG. 7).

### Methods

### ADP/ATP Exchange Analysis:

Essentially, the ADP/ATP exchange through mouse Ant are measured using the method described in Palmieri 1995, Dolce 2005 and Haferkamp 2002. Dr. Brian Cain, the co-investigator of the project is an expert in mitochondrial membrane biochemistry, and has extensive experience in all aspects of the proposed experiments.

(a) DNA Constructs for Expression of Ant in E. coli

The expression plasmids (pET21b, Novagen) encoding the recombinant mouse Ant1, Ant2, and Ant4 proteins areconstructed as follows: the cDNA coding the entire Ants are generated by PCR from first-strand mouse cDNAs. Sense primers incorporating a Sal I restriction site and antisense primers with Not I are used for the PCR reaction. The obtained PCR products are purified, subcloned into the pCR2.1 Topo vector and checked by sequencing both strands by chain-termination reaction. For the construction of the E. coli expression plasmids, the Sal I and Not I DNA inserts of the pCR2.1-Topo are introduced in-frame into the corresponding restriction sites of the isopropyl thio-β-D-galactoside (IPTG)-inducible T7-RNA polymerase bacterial expression vector pET21b. Transformations of E. coli are carried out according to standard protocols.

### (b) Preparation of Recombinant Ant Proteins

The mouse Ant-expression vectors are expressed as inclusion bodies in E. coli BL-21 CodonPlus (DE3)-RIL. Ant(s) are expressed by IPTG induction in 1 L cultures at a final concentration of 1 mM for 3-4 hours. Inclusion bodies are isolated and Ant(s) are purified by centrifugation and washing steps as described (Palmieri, 1995, 2001). Briefly, inclusion bodies are purified on a sucrose density gradient, washed at 4° C. with TE buffer (10mM Tris-HCl, 1 mM EDTA pH 6.5), then twice with a buffer containing Triton X-114 (3%, w/v), 1 mM EDTA and 10 mM PIPES-KOH (pH 6.5), and once again with TE buffer. The recombinant protein is solubilized in 1.9% sarkosyl (w/v), and a small residue is removed by centrifugation (258,000×g, 1 h).

### (c) Reconstitution of Ant Into Liposomes

The recombinant protein in sarkosyl is reconstituted into liposomes in the presence of substrates. To prepare liposomes, we dissolve egg yolk phosphatidylcholine and cardiolipin (1.14 mg/ml) in chloroform. The mixtures are dried in vacuum overnight. The dried lipid is dissolved in buffer (20 mM PIPES, pH 6.8, 1 mM EDTA) 10% (w/v) and vortexed intensely. After three times of freeze (liquid nitrogen) and thaw (37° C. water bath) cycle, the mixture is passed through a 100-nm polycarbonate membrane filter 13 times. We reconstitute proteoliposome by detergent removal method. Briefly, solubilized proteins are diluted in buffer (10-20 mM PIPES-KOH/NaOH pH 6.5-8.0, 1 mM EDTA) and mixed as follows in this order: 200-400ul of purified protein. (0.1-15 ug). with substrate (ADP or ATP), 90 ul of 10% Triton X-114 (total detergent:lipid ratio=1:1.6), and 100 ul of 10% Liposome (prepared above). After vortexing well, the mixture is passed through resin column (packed with 2 ml of Amberlite XAD-2) 13 times. External substrate are removed from proteoliposomes on Sephadex G-75.

(d) Transport Assay and Kinetics

Transport at 25° C. is started by adding various concentrations (18-2000DM) of [¹⁴C]ADP or [¹⁴C]ATP to proteoliposomes. The reaction is terminated in one to 30 min by addition of 30 mM pyridoxal 5′-phosphate and 20 mM bathophenanthroline. The proteoliposomes are separated by Sephadex G-50 to remove external radioactivity. The eluted proteoliposomes are mixed with 4 ml scintillation mixture with a high capacity of solubilizing water. The radioactivity is counted as dpm by liquid scintillation equipment. The radioactivity that has entered the active volume of proteoliposomes by carrier-mediated exchange (dpm_{active}) is obtained by subtracting the dpm of the controls (with the inhibitor, bongkrekic acid added together with the substrate) from the dpm of the samples. The nanomoles of substrate taken up are calculated as dpm_{active}/specific radioactivity, where specific radioactivity is given by the ratio between added dpm and added nanomoles of substrate. The transport activity is finally expressed as nmoles substrate/mg protein per time. For kinetic measurement, initial transport rates (nmol/mg protein per min) can be obtained by measuring transport in short times (1 to 2 min). The Vmax & Km will be calculated by following the Chemical Genomics Center (NCGC) guidance for enzyme assay (http://www.ncgc.nih.gov/guidance/section4.html#inhibition-constant).

### (e) Inhibition by Small Compounds:

In the transport assay above, various concentrations of small compounds are added with the radio-labeled substrate, and the transported radioactivity is measured as described above. Bongkrekic acid is used as a control, as it blocks transport for all Ants (Dolce 2005). IC₅₀ values of lead compounds for Ant1, 2 and 4 are calculated.

### Example 4

### Effects of Compounds on Male Meiosis in vivo

Compounds demonstrating a selective inhibition of Ant4 over other Ants are tested for inhibition of male meiosis and fertility in vivo. Initially, lead compounds or vehicle alone are intratesticularly (i.t.) injected into male C57B6 mice (8 weeks old) at a dose to be optimized (FIG. 8). Since the process of spermatocyte development takes ˜14 days in mice, we sacrifice the mice 14 days after administration, and the effects of the compounds on male meiosis are evaluated. Alternatively, lead compounds or vehicle alone are intraperitoneally (i.p.) injected into the mice at a dose and timing to be optimized. A schedule for the i.p. experiment is also shown in FIG. 8.

### Methods

### Testicular Histology:

Testicular weight and histology are analyzed as described above (FIG. 9) (Brower 2007).

### Meiotic Arrest:

Meiotic arrest is examined using Sycpp3 staining in seminiferous tubule sections (FIG. 10) and germ cell chromosomal spread analysis (FIG. 11) as described above.

### Apoptosis of Germ Cells:

Apoptosis of male germ cells is analyzed as described above (FIG. 11) (Brower 2007).

### Male Fertility:

After determining the optimal doses of the drugs in the experiments designed above, we test the effects of the drugs on male fertility in mice. Here, we treat the mice with compounds for a longer term (8 weeks) by continuing the i.p. injections. After 4 weeks, the male mice are cohabitated with a female C57B6 mouse until a copulation plug is observed in the female. The female with the plug is exchanged for a new female C57B6 mouse. The procedure is repeated at least three times at 4, 6 and 8 weeks respectively. The number of progeny from each is counted.

### Reversibility:

Spermatogonial stem cells appear to survive in adult Ant4-deficient mice, therefore, it is expected that Ant4 inhibition by chemicals will not lead to permanent sterility. Reversible fertility is examined by withdrawing the drugs after 8 weeks in vivo, and by repeating male/female cohabitation procedures as described above.

### Adverse Effects:

While we anticipate little distress with these experiments, the mice are monitored twice daily during and after injections for any adverse effects. The injection site is observed for leakage and palpated to detect any injection-site tumors, though this is also mediated by alternating injection locations. We will also look for signs of lethargy, lowered body temperature and weight loss. The animals should be able to carry on normal daily functions such as eating, drinking, etc. We act in accordance with veterinary recommendations on all adverse effects. No pain, distress or discomfort is anticipated for the euthanasia procedure.

### Example 5

### ANT4 Profile

### (1) Ant4 is Expressed Exclusively in Male Germ Cells

In order to determine the exact expression profile of the Ant4 gene in testis, we initially performed an immunohistochemical analysis using specific antibodies raised against mouse Ant4 or human ANT4 (Brower 2007). FIG. 12A and 12B indicate that the highest levels of Ant4 protein are found within primary spermatocytes, whereas sperrnatogonial cells express a lower level. Importantly, Sertoli cells or other somatic interstitial cells did not express ANT4. In order to further define the stage specific-expression pattern of Ant4 in male germ cells, Ant4 mRNA levels in separated spermatogenic cell types of mouse, were analyzed using real-time RT-PCR (FIG. 12C). Ant4 transcript levels began to increase during the transition of premeiotic type B spermatogonia to the early stages of meiosis as represented by preleptotene spermatocytes (PL). The transcript levels of Ant4 continued to increase through the leptotene (L) and zygotene (Z) spermatocyte stages, peaking in early pachytene (EP) spermatocytes. Ant4 transcript levels then began to decrease in late pachytene (LP) spermatocytes and in later round spermatids (RS). Thus, high levels of Ant4 expression are likely associated with entry of male germ cells into meiosis. In contrast, the fraction enriched in. Sertoli cells (JS) expressed a very low level of Ant4. We also confirmed here, by real time RT-PCR, that the Ant4 transcript is very low or undetectable in somatic organs and ovary.

Using the same RNA samples prepared for the study above, we also investigated the expression pattern of the Ant2 gene in various organs and spermatogenic cell types (FIG. 12C). Of interest, the expression profile of Ant2 in mice was reciprocal to that of Ant4. The Ant2 transcript was high in somatic organs, but relatively low in whole testis and almost completely undetectable in testicular germ cells.

### (2) Ant4 is Essential for Spermatogenesis and Male Fertility

To investigate the in vivo function of Ant4, we generated Ant4-deficient mice by homologous recombination in embryonic stem (ES) cells (Brower 2007). The. Ant4^{-/-} mice were viable and exhibited apparently normal development. The interbreeding of Ant4^{+/-} mice produced offspring of normal litter size, and conformed to the Mendelian ratios of Ant4^{+/+}, Ant4^{+/-} and Ant4^{-/-} inheritance. In contrast to the similar body sizes between the wild type and mutant mice (data not shown), the testes of Ant4^{-/-} adults were smaller than those of Ant4^{+/+} and Ant4^{+/-} adults (FIG. 9A). Testes from 6-week-old Ant4^{-/-} males were approximately one-third the weight of those from control males. Closer examination of testicular development revealed similar growth patterns of the testes until approximately 17 days after birth, suggesting normal growth at earlier spermatogenic stages (FIG. 9B). Subsequent development was impaired in Ant4^{-/-} testis. Histological analysis of Ant4-deficient testis demonstrated clear morphological aberrations in the process of spermatogenesis as evident by the severe reduction of spermatocytes and absence of spermatids and sperm (FIG. 9C). In summary, these data indicate that Ant4 is essential for murine spermatogenesis and male fertility.

### (3) Ant4-Deficient Male Mice Exhibit Meiotic Arrest.

To investigate the stage of arrest of Ant4-deficient spermatogenic cells, we initially stained testicular cross-sections with an anti-synaptonemal complex protein 3 (Sycp3) antibody (FIG. 10) (Brower et al., in preparation). Sycp3 mediates the pairing and synapsis of homologous chromosomes during meiosis I and thus is commonly utilized to demarcate the chromosomes during meiosis I. Upon analysis with Sycp3, the seminiferous epithelium of Ant4-deficient mice showed an increased proportion of leptotene-like spermatocytes, as determined by the lesser degree of chromosomal condensation. The Ant4-deficient spermatocytic compartment also contained zygotene and pachytene-like cells (i.e. highly condensed chromosomes visualized by Sycp3), however the proportion of these cell types were decreased in comparison to controls (FIG. 10).

To further determine the stage of arrest of Ant4-deficient meiotic spermatocytes, we have developed a chromosomal spread technique in the lab, by adapting the protocol from Nickerson et al. 2007, and stained the chromosomes with Sycp3, γH2AX and DAPI (FIG. 11A, 11B) (Brower et al., in preparation). Phosphorylated H2AX (γH2AX) is a histone variant known to associate with double strand breaks and stain leptotene chromosomes diffusely. γH2AX is also known to associate with the inactivated sex chromosomes during meiosis I, specifically during the zygotene through diplotene stages of prophase I. γH2AX is implicated to have a role in conferring the heterochromatic transformation of the X and Y chromosomes that occurs during meiosis I. Briefly, testes were dissected from adult mice (6 weeks) and decapsulated. Tunica albuginea and extratubular tissue were removed by rinsing the seminiferous tubules in PBS. Tubules were then placed in a hypotonic extraction buffer for 30 min. One-inch lengths of tubules were placed in 20 μl of sucrose solution (sucrose, 100 mM) and torn, into small pieces with fine forceps. The volume was then increased to 40 μl with sucrose solution and pipetted to give a cloudy suspension, which was spread onto two slides covered in formaldehyde solution (1% formaldehyde, 0.15% Triton X-100, adjusted to Ph 9.2 with sodium borate). Slides were then air-dried for 2 hr and used immediately or stored at −20/−80° C. For immunostaining, slides were rinsed for 5 min in PBS and then incubated for 30 min in wash/dilution buffer (3% BSA, 0.5% Triton X100 in PBS). Antibodies were diluted in wash/dilution buffer and incubated at 4° C. overnight. Following three 3 min washes in PBS, fluorescent conjugated secondary antibodies were added and incubated for 45 min at room temperature in the dark. Slides were then washed in PBS, stained and mounted with Vectashield (Vector Laboratories, Inc. Burlingame, Calif.) containing DAPI.

Sycp3 and γH2AX localization allowed us to determine exactly which stages of meiotic prophase I were absent in the Ant4-deficient mice in comparison to controls. FIG. 11A, 11B illustrates the meiotic stages found in wild type and Ant4-deficient mice. FIG. 11C illustrates a quantitative comparison of Ant4 wild-type and Ant4-deficient testicular preparations. In summary, we found a significantly increased percentage of leptotene spermatocytes, a severe reduction in the percentage of pachytene spermatocytes and complete absence of diplotene spermatocytes in the Ant^{-/-} testes. In addition, the majority of pachytene spermatocytes found in the Ant4^{-/-} testes demonstrated abnormalities including less condensed chromatin and association of γH2AX outside of the sex chromosomes.

### (4) Ant4-Deficient Male Mice Exhibit Increased Levels of Apoptosis

TUNEL labeling and caspase-3 staining were utilized to analyze the apoptotic profile of adult (6 wks) Ant4^{-/-} testis in comparison to controls (FIG. 13). The testis of Ant4-deficient mice exhibited increased levels of TUNEL-positive cells within the seminiferous tubules as compared to controls (FIG. 13A). Upon closer examination, the majority of the TUNEL-positive cells within the seminiferous tubules of Ant4^{-/-} mice appeared to be spermatocytes based upon cellular morphology and positioning within the seminiferous epithelium (FIG. 13A bottom panel). We also utilized caspase-3 staining within the testis to confirm the differential apoptotic profiles present between Ant4-deficient testis and controls (FIG. 13B). Taken together, these results suggest that the Ant4^{-/-} testis contains a significantly higher number of apoptotic cells than controls, and the majority of these cells appear to be early spermatocytes.

Taken together, these data elucidated the indispensable role of Ant4 in murine male meiosis. Considering the unique conservation and chromosomal location of the Ant family of genes in mammals, the Ant4 gene may have arisen in mammalian ancestors and been conserved in mammals to serve as the essential mitochondrial ADP/ATP carrier during spermatogenesis where the sex chromosome-linked Ant2 gene is inactivated.

### (5) Ant4 Expression in Sperm Mitochondria

Since all other Ants are strictly localized to the inner mitochondrial membrane (see Background), we speculated that Ant4 would also localize to mitochondria. This speculation was supported by a punctate Ant4 staining pattern in the cytoplasm of spermatocytes (FIG. 12) and its co-localization with Hsp60. However, a previous paper demonstrated that Ant4 was present and accumulated in the fibrous sheath of the human sperm flagellar principal piece (Kim 2007). In order to examine whether this was truly the case, we carried out immunostaining of mouse and human sperm using our antibodies raised against mouse and human Ant4, respectively. Importantly, our affinity-purified antibodies stain Ant4 specifically, judging by immunoblot analysis (data not shown). Of interest, our immunocytochemistry data demonstrated that the mid-piece of mouse sperm was predominantly stained with the antibodies (FIG. 5). Localization of the protein in mitochondria was confirmed by co-staining with MitoTracker (Invitrogen) (data not shown). Mid-piece-dominant staining was also demonstrated in human sperm (data not shown). No significant staining was detected in the flagellar principal piece in either mouse or human sperm. These data question the previous study by Kim et al (2007). It should be noted that the polyclonal antibodies they used for immunostaining in their study were not affinity-purified. Based on the highly conserved transmembrane domains in Ant4 and its mitochondrial localization when expressed in cell lines, we believe that it is very unlikely that Ant4 localizes outside of the mitochondrial membrane.

We found Ant4-deficiency resulted in the complete absence of diplotene spermatocytes, whereas the wild-type mice display an apparently normal proportion of diplotene spermatocytes. Further, disruption of Ant4 resulted in a severe reduction in the percentage of pachytene spermatocytes in comparison to wild-type controls. We also found an accumulation of leptotene spermatocytes upon Ant4 disruption without a significant change in the proportion of zygotene spermatocytes. These data taken together indicate that Ant4 deficiency impairs the progression of male meiosis, and that this impairment becomes most evident at the transition from leptotene to zygotene stage.

### REFERENCES

Amiri H, Karlberg O, Andersson SGV (2003) Deep origin of plastid/parasite ATP/ADP translocases. J Mol Evol 56, 137-50
Amory J K, Page S T, Bremner WJ (2006) Drug insight: recent advances in male hormonal contraception. Nat Clin Practice 2, 32-41
Bamber L, Harding M, Monné M, Slotboom D J, Kunji E R (2007) The yeast mitochondrial ADP/ATP carrier functions as a monomer in mitochondrial membranes. Proc Natl Acad Sci USA 104, 10830-4
Belzacq A S, Vieira H L, Kroemer G, Brenner C (2002) The adenine nucleotide translocator in apoptosis. Biochimie 84, 167-76
Besculides M, Laraque F (2004) Unintended pregnancy among the urban poor. J Urban Health 81, 340-348
Brower J V, Rodić N, Seki T, Jorgensen M, Fliess N, Yachnis A T, McCarrey J, Oh S P, Terada N (2007) Evolutionarily conserved mammalian adenine nucleotide translocase 4 is essential for spermatogenesis. J Biol Chem 282, 29658-66
Ceci J D (1994) Mouse chromosome 8. Mamm Genome 5, S124-38
Dammkoehler R A, Karasek S F, Shands E F B, Marshall G R (1989) Constrained Search of Conformational Hyperspace. J. Comp.-Aided Mol. Des. 3, 3-21
Dolce V, Scarcia P, Iacopetta D, Palmieri F (2005) A fourth ADP/ATP carrier isoform in man: identification, bacterial expression, functional characterization and tissue distribution. FEBS Lett 579, 633-7
Ellison J W, Salido E C, Shapiro L J (1996) Genetic mapping of the adenine nucleotide translocase-2 gene (Ant2) to the mouse proximal X chromosome. Genomics 36, 369-71
Fiore C, Trézéguet V, Le Saux A, Roux P, Schwimmer C, Dianoux A C, Noél F, Lauquin G J, Brandolin G, Vignais P V (1998) The mitochondrial ADP/ATP carrier: structural, physiological and pathological aspects. Biochimie 80, 137-50
Glasier A F, Anakwe R, Everington D, Martin C W, van der Spuy Z, Cheng L, Ho P C, Anderson R A (2000) Would women trust their partners to use a male pill? Human Reprod 15, 646-649
Graham B H, Waymire K G, Cottrell B, Trounce I A, MacGregor G R, Wallace D C (1997) A mouse model for mitochondrial myopathy and cardiomyopathy resulting from a deficiency in the heart/muscle isoform of the adenine nucleotide translocator. Nat Genet 16, 226-34
Hackenberg H, Klingenberg M (1980) Molecular weight and hydrodynamic parameters of the adenosine 5′-diphosphate-adenosine 5′-triphosphate carrier in Triton X-100. Biochemistry 19, 548-55
Haferkamp I, Hackstein J H P, Voncken F G, Schmit G, Tjaden J (2002) Functional integration of mitochondrial and hydrogenosomal ADP/ATP carriers in the Escherichia coli membrane reveals different biochemical characteristics for plants, mammals and anaerobic chytrids. Eur J Biochem 269, 3172-81
Halestrap A P, Woodfield K Y, Connem C P. (1997) Oxidative stress, thiol reagents, and membrane potential modulate the mitochondrial permeability transition by affecting nucleotide binding to the adenine nucleotide translocase. J Biol Chem 272, 3346-54
Heinemann K, Saad F, Wiesernes M, White S, Heinemann L (2005) Attitudes toward male fertility control: results of a multinational survey on four continents. Hum Reprod 20, 549-556
Hernandez Prada J A, Ferreira A J, Katovich M J, Shenoy V, Qi Y, Santos R A, Castellano R K, Lampkins A J, Gubala V, Ostrov D A, Raizada M K (2008) Structure-based identification of small-molecule angiotensin-converting enzyme 2 activators as novel antihypertensive agents. Hypertension 51, 1312-7
Henshaw S K (1998) Unintended pregnancy in the US. Fam Plann Perspect 30, 24-29
Ho C M W, Marshall G R (1990) CAVITY SEARCH: An Algorithm for the Isolation and Display of Cavity-like Binding Regions. J Comp-Aided Mol Des 4, 337-354.
Ho C M W, Marshall G R (1993) SPLICE: A Program to Assemble Partial Query Solutions from Three-Dimensional Database Searches into Novel Ligands. J Comp-Aided Mol Des 7, 623-647.
Ho C M W, Marshall G R (1995) DBMAKER: A Set of Programs to Generate Three-Dimensional Databases Based upon User-Specified Criteria. J Comp-Aided Mol Des 9, 65-86.
Huizing M, Ruitenbeek W, Van den Heuvel L P, Dolce V, Iacobazzi V, Smeitinck J A M, Palmieri F, Trijbels J M (1998) Human mitochondrial transmembrane metabolite carriers: tissue distribution and its implications for mitochondrial disorders. J of Bioenerg and Biomembr 30, 277-84
Huentelman M J, Zubcevic J, Hernandez Prada J A, Xiao X, Dimitrov D S, Raizada M K, Ostrov D A (2004) Structure-based discovery of a novel angiotensin-converting enzyme 2 inhibitor Hypertension 44, 903-6
Jang J Y, Choi Y, Jeon Y K, Kim C W (2008) Suppression of adenine nucleotide translocase-2 by vector-based siRNA in human breast cancer cell induces apoptosis and inhibits tumor growth in vitro and in vivo. Breast Cancer Research 10, R11, Epub 2008 Feb. 12
Kasahara M, Hinkle P C (1976) Reconstitution of D-glucose transport catalyzed by a protein fraction from human erythrocytes in sonicated liposomes. Proc Nat Acad Sci USA 73, 396-400
Kim Y H, Haidl G, Schaefer M, Egner U, Herr J C (2007) Compartmentalization of a unique ADP/ATP carrier protein SFEC (sperm flagellar energy carrier, AAC4) with glycolytic enzymes in the fibrous sheath of the human sperm flagellar principal piece. Dev Biol 302, 463-76.
Klingenberg M (1989) Molecular aspects of the adenine nucleotide carrier from mitochondria. Arch Biochem Biophys 270,1-14
Kokoszka J E, Waymire K G, Levy S E, Sligh J E, Cai J, Jones D P, MacGregor G R, Wallace D C (2004) The ADP/ATP translocator is not essential for the mitochondrial permeability transition pore. Nature 427, 461-5
Kolarov J, Kolarova N, Nelson N (1990) A third ADP/ATP translocator gene in yeast. J Biol Chem 265, 12711-6
Kusakabe H, Kamiguchi Y (2004) Chromosome analysis of mouse zygotes after injecting oocytes with spermatozoa treated in vitro with green tea catechin, (-)-epigallocatechin gallate (EGCG). Mutation Research 564, 195-200.
Lunardi J, Hurko O, Engel W K, Attardi G (1992) The multiple ADP/ATP translocase genes are differentially expressed during human muscle development. J Biol Chem 267,15267-70
Martin C W, Anderson R A, Cheng L, Ho P C, van der Spuy Z, Smith K B, Glasier Everington D, Baird D T (2000) Potential impact of hormonal male contraception: cross-cultural implications for development of novel male preparations. Human Reprod 15, 637-645
Miki K, Qu W, Goulding E H, Willis W D, Bunch D O, Strader L F, Perreault S D, Eddy E M, O'Brien D A (2004) Glyceraldehyde 3-phosphate dehydrogenase-S, a sperm-specific glycolytic enzyme, is required for sperm motility and male fertility. Proc Nat Acad Sci USA 101, 16501-6
Minamino T, Yujiri T, Papst P J, Chan E D, Johnson G L, Terada N (1999) MEKK1 suppresses oxidative stress-induced apoptosis of embryonic stem cell-derived cardiac myocytes. Proc Natl Acad Sci USA. 96, 15127-32
Nelson D R, Felix C M, Swanson J M (1998) Highly conserved charge-pair networks in the mitochondrial carrier family. J Mol Biol 277, 285-308
Nickerson H D, Joshi A, Wolgemuth D J (2007) Cyclin A1-deficient mice lack histone H3 serine 10 phosphorylation and exhibit altered aurora B dynamics in late prophase of male meiosis. Dev Biol 306, 725-735
Nicolli A, Basso E, Petronilli V, Wenger R M, Bernardi P (1996) Interactions of cyclophilin with the mitochondrial inner membrane and regulation of the permeability transition pore, and cyclosporin A-sensitive channel. J Biol Chem 271,2185-92
Oh S-H, Miyoshi K, Funahashi H (1998) Rat oocytes fertilized in modified rat 1-cell embryo culture medium containing a high sodium chloride concentration and bovine serum albumin maintain developmental ability to the blastocyst stage. Biol of Reproduction 59, 884-889.
Oka M, Meacham A M, Hamazaki T, Rodic N, Chang L-J, Terada N (2005) De novo DNA methyltransferases Dnmt3a and Dnmt3b primarily mediate the cytotoxic effect of 5-aza-2′-deoxycytidine. Oncogene 24, 3091-9
Ostrov D A, Hernández Prada J A, Corsino P E, Finton K A, Le N, Rowe T C (2007) Discovery of novel DNA gyrase inhibitors by high-throughput virtual screening Antimicrob Agents Chemother 51, 3688-98
Palmieri F, Indiveri C, Bisaccia F, Iacobazzi V (1995) Mitochondrial metabolite carrier proteins: purification, reconstitution and transport studies. Methods Enzymol 260, 349-69
Palmieri L, Lasorsa F M, Vozza A, Agrimi G, Fiermonte G, Runswick M J, Walker J E, Palmieri F. (2000) Identification and functions of new transporters in yeast mitochondria. Biochim Biophys Acta 1459, 363-9
Palmieri L, Pardo B, Lasorsa F M, del Arco A, Kobayashi K, lijima M, Runswick M J, Walker J E, Saheki T, Satrustegui J, Palmieri F (2001) Citrin and aralar 1 are Ca²⁺-stimulated aspartate/glutamate transporters in mitochondria. EMBO J 20, 5060-9
Pebay-Peyroula E, Dahout-Gonzalez C, Kahn R, Trézéguet V, Lauquin G J, Brandolin G (2003) Structure of mitochondrial ADP/ATP carrier in complex with carboxyatractyloside. Nature 426, 39-44
Piccinino L J, Mosher W D (1998) Trends in contraceptive use in the United States: 1982-1995. Fam Plan Perspect 30, 4-10
Rodic N, Oka M, Hamazaki T, Murawski M R, Jorgensen M, Maatouk D M, Resnick JL, Li E, Terada N (2005) DNA methylation is required for silencing of Ant4, an adenine nucleotide translocase selectively expressed in mouse embryonic stem cells and germ cells. Stem Cells 23, 1314-23
Sabova L, Zeman I, Supek F, Kolarov J (1993) Transcriptional control of AAC3 gene encoding mitochondrial ADP/ATP translocator in Saccharomyces cerevisiae by oxygen, heme and ROX1 factor. Eur J Biochem 213, 547-53
Sandberg E M, Ma X, He K, Frank S J, Ostrov D A, Sayeski P P (2005) Identification of 1,2,3,4,5,6-hexabromocyclohexane as a small molecule inhibitor of jak2 tyrosine kinase autophosphorylation J Med Chem 48, 2526-33
Stepien G, Torroni A, Chung A B, Hodge J A, Wallace D C (1992) Differential expression of adenine nucleotide translocator isoforms in mammalian tissues and during muscle cell differentiation. J Biol Chem 267, 14592-7
Terada N, Franklin RA, Lucas J J, Blenis J, Gelfand E W (1993) Failure of rapamycin to block proliferation once resting cells have entered cell cycle despite inactivation of p70 S6 kinase. J Biol Chem 268, 12062-8
Van Dop C, Hutson S M, Lardy H A (1977) Pyruvate metabolism in bovine epididymal spermatozoa. J Biol Chem 252, 1303-8
Wu G-J, Ding D-C, Chen I-G, Yuang Y-C (2004) Less NO production and better motion parameter in human sperm by swim-up processing. Arch Androl 50, 373-377
Zoratti M, Szabo I (1995) The mitochondrial permeability transition. Biochim Biophys Acta 1241, 139-76

The disclosures of each and every patent, patent application and publication cited herein are hereby incorporated herein by reference in their entirety.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

Although the invention has been disclosed with reference to specific embodiments, it is apparent that other embodiments and variations of the invention may be devised by others skilled in the art without departing from the true spirit and scope of the invention. The sentences are intended to be construed to include all such embodiments and equivalent variations.

1. A method of treating a subject suffering from or susceptible to a cell proliferation related disorder or disease, the method comprising administering to a subject in need thereof a therapeutically effective amount of an adenine nucleotide translocase 4 (ANT4) inhibitor, to thereby treat the subject suffering from or susceptible to a cell proliferation related disorder or disease.

2. The method of claim 1, wherein the ANT4 inhibitor is a selective ANT inhibitor.

3. The method of claim 1, wherein the ANT4 inhibitor is selected from
a compound of Table 1, or a pharmaceutically acceptable salt or prodrug thereof.

4. A method for identifying a compound that inhibits ANT4 activity, the method comprising:
a) obtaining a crystal structure of ANT4 or obtaining information relating to the crystal structure of ANT4, and
b) modeling a test compound into or on the crystal structure coordinates to determine whether the compound binds to ANT4.

5. The method of claim 4, wherein the step of modeling comprises modeling or determining the ability of the compound to bind to or associate with a binding pocket defined by structure coordinates of one or more ANT4 amino acid residues selected from amino acids 62-63 and 257-261 of mouse Ant4.

6-10. (canceled)

11. A pharmaceutical composition comprising a compound of Table 1, or a pharmaceutically acceptable salt or prodrug thereof, together with a pharmaceutically acceptable carrier.

12-13. (canceled)

14. A method of inducing contraception in a male subject, the method comprising inhibiting the activity of adenine nucleotide translocase 4 (ANT4) in the subject.

15. The method of claim 14, wherein the inhibition is such that the subject has reduced sperm production compared to without inhibition of ANT4.

16-17. (canceled)

18. A method of inducing contraception or reducing or eliminating male meiotic spermatocytes selectively without damaging other cell types in a subject, the method comprising administering to the subject a compound that inhibits adenine nucleotide translocase 4 (ANT4).

19. The method of claim 18, wherein the subject is a mammal.

20. The method of claim 18, wherein the activity of adenine nucleotide translocase 4 (ANT4) in the cell is inhibited by contacting the cell with a selective inhibitor of ANT4.

21-22. (canceled)

23. The method of claim 4, comprising using the atomic coordinates of an amino acid sequence comprising ANT4 amino acid residues 62-63 and 257-261 of mouse Ant4.

24. The method of claim 2, wherein the ANT4 inhibitor compound is demonstrated to bind to ANT4 at an amino acid sequence comprising ANT4 amino acid residues 62-63 and 257-261 of mouse Ant4.

25. The method of claim 1, wherein the subject is identified as in need of treatment by administration of an ANT4 inhibitor compound.

26. The method of claim 1, wherein the subject is identified as in need of treatment by administration of a ANT4 inhibitor compound that is demonstrated to bind to ANT4 at an amino acid sequence comprising ANT4 amino acid residues 62-63 and 257-261 of mouse Ant4.

27. (canceled)

28. The method of claim 18, wherein the ANT4 inhibitor compound is selected from a compound of Table 1, or a pharmaceutically acceptable salt or prodrug thereof.

29. The method of claim 1, wherein the cell proliferation related disorder or disease is a type III germ cell tumor.

30. The method of claim 18, wherein the ANT4 inhibitor compound impairs transition during spermatogenesis from the leptotene stage to the zygotene stage in the progression of male meiosis.
